SEQUENCE LISTING (66 sequences)

SEQ ID NO: 1 - protein, 28 aa, Bovine
  VARIANT 15, 17, 18, 19: Phosphorylated amino acids
RELEELNVPGEIVESLSSSEESITRINK

SEQ ID NO: 2 - protein, 29 aa, Bovine
  VARIANT 15,17,18,19: Phosphorylated amino acids
RELEELNVPGEIVESLSSSEESITRINKK

SEQ ID NO: 3 - protein, 16 aa, Bovine
  VARIANT 3: Phosphorylated amino acid
FQSEEQQQTEDELQDK

SEQ ID NO: 4 - protein, 49 aa, Bovine
IHPFAQTQSLVYPFPGPIPNSLPQNIPPLTQTPVVVPPFLQPEVMGVSK

SEQ ID NO: 5 - protein, 6 aa, Bovine
EAMAPK

SEQ ID NO: 6 - protein, 6 aa, Bovine
EMPFPK

SEQ ID NO: 7 - protein, 56 aa, Bovine
YPVEPFTESQSLTLTDVENLHLPLPLLQSWMHQPHQPLPPTVMFPPQSVLSLSQSK

SEQ ID NO: 8 - protein, 7 aa, Bovine
VLPVPQK

SEQ ID NO: 9 - protein, 33 aa, Bovine
AVPYPQRDMPIQAFLLYQEPVLGPVRGPFPIIV

SEQ ID NO: 10 - protein, 25 aa, Bovine
  VARIANT 15,17,18,19: Phosphorylated amino acids
RELEELNVPGEIVESLSSSEESITR

SEQ ID NO: 11 - protein, 4 aa, Human
LEEK

SEQ ID NO: 12 - protein, 8 aa, Human
VCQGTSNK

SEQ ID NO: 13 - protein, 43 aa, Human
  VARIANT 19: Glycosylated amino acid
LTQLGTFEDHFLSLQRMFNNCEVVLGNLEITYVQRNYDLSFLK

SEQ ID NO: 14 - protein, 49 aa, Human
  VARIANT 48: Glycosylated amino acid
TIQEVAGYVLIALNTVERIPLENLQIIRGNMYYENSYALAVLSNYDANK

SEQ ID NO: 15 - protein, 4 aa, Human
TGLK

SEQ ID NO: 16 - protein, 56 aa, Human
  VARIANT 42: Glycosylated amino acid
ELPMRNLQEILHGAVRFSNNPALCNVESIQWRDIVSSDFLSNMSMDFQNHLGSCQK

SEQ ID NO: 17 - protein, 20 aa, Human
  VARIANT 7: Glycosylated amino acid
CDPSCPNGSCWGAGEENCQK

SEQ ID NO: 18 - protein, 14 aa, Human
IICAQQCSGRCRGK

SEQ ID NO: 19 - protein, 27 aa, Human
SPSDCCHNQCAAGCTGPRESDCLVCRK

SEQ ID NO: 20 - protein, 8 aa, Human
FRDEATCK

SEQ ID NO: 21 - protein, 23 aa, Human
DTCPPLMLYNPTTYQMDVNPEGK

SEQ ID NO: 22 - protein, 9 aa, Human
YSFGATCVK

SEQ ID NO: 23 - protein, 32 aa, Human
KCPRNYVVTDHGSCVRACGADSYEMEEDGVRK

SEQ ID NO: 24 - protein, 31 aa, Human
CPRNYVVTDHGSCVRACGADSYEMEEDGVRK

SEQ ID NO: 25 - protein, 7 aa, Human
CEGPCRK

SEQ ID NO: 26 - protein, 11 aa, Human
VCNGIGIGEFK

SEQ ID NO: 27 - protein, 11 aa, Human
DSLSINATNIK

SEQ ID NO: 28 - protein, 36 aa, Human
  VARIANT 1: Glycosylated amino acid
NCTSISGDLHILPVAFRGDSFTHTPPLDPQELDILK

SEQ ID NO: 29 - protein, 32 aa, Human
  VARIANT 14: Glycosylated amino acid
EITGFLLIQAWPENRTDLHAFENLEIIRGRTK

SEQ ID NO: 30 - protein, 23 aa, Human
  VARIANT 13: Glycosylated amino acid
QHGQFSLAVVSLNITSLGLRSLK

SEQ ID NO: 31 - protein, 13 aa, Human
EISDGDVIISGNK

SEQ ID NO: 32 - protein, 11 aa, Human
NLCYANTINWK

SEQ ID NO: 33 - protein, 9 aa, Human
KLFGTSGQK

SEQ ID NO: 34 - protein, 8 aa, Human
LFGTSGQK

SEQ ID NO: 35 - protein, 11 aa, Human
IISNRGENSCK

SEQ ID NO: 36 - protein, 38 aa, Human
  VARIANT 28: Glycosylated amino acid
ATGQVCHALCSPEGCWGPEPRDCVSCRNVSRGRECVDK

SEQ ID NO: 37 - protein, 55 aa, Human
  VARIANT 30: Glycosylated amino acid
CNLLEGEPREFVENSECIQCHPECLPQAMNITCTGRGPDNCIQCAHYIDGPHCVK

SEQ ID NO: 38 - protein, 16 aa, Human
  VARIANT 10: Glycosylated amino acid
TCPAGVMGENNTLVWK

SEQ ID NO: 39 - protein, 33 aa, Human
  VARIANT 14: Glycosylated amino acid
YADAGHVCHLCHPNCTYGCTGPGLEGCPTNGPK

SEQ ID NO: 40 - protein, 34 aa, Human
IPSIATGMVGALLLLLVVALGIGLFMRRRHIVRK

SEQ ID NO: 41 - protein, 32 aa, Human
  VARIANT 17: Phosphorylated amino acid
RTLRRLLQERELVEPLTPSGEAPNQALLRILK

SEQ ID NO: 42 - protein, 31 aa, Human
ETEFKKIKVLGSGAFGTVYKVKGLWIPEGEK

SEQ ID NO: 43 - protein, 12 aa, Human
VLGSGAFGTVYK

SEQ ID NO: 44 - protein, 9 aa, Human
GLWIPEGEK

SEQ ID NO: 45 - protein, 8 aa, Human
VKIPVAIK

SEQ ID NO: 46 - protein, 6 aa, Human
IPVAIK

SEQ ID NO: 47 - protein, 9 aa, Human
ELREATSPK

SEQ ID NO: 48 - protein, 52 aa, Human
ANKEILDEAYVMASVDNPHVCRLLGICLTSTVQLITQLMPFGCLLDYVREHK

SEQ ID NO: 49 - protein, 49 aa, Human
EILDEAYVMASVDNPHVCRLLGICLTSTVQLITQLMPFGCLLDYVREHK

SEQ ID NO: 50 - protein, 17 aa, Human
DNIGSQYLLNWCVQIAK

SEQ ID NO: 51 - protein, 23 aa, Human
GMNYLEDRRLVHRDLAARNVLVK

SEQ ID NO: 52 - protein, 6 aa, Human
TPQHVK

SEQ ID NO: 53 - protein, 8 aa, Human
ITDFGLAK

SEQ ID NO: 54 - protein, 7 aa, Human
LLGAEEK

SEQ ID NO: 55 - protein, 12 aa, Human
EYHAEGGKVPIK

SEQ ID NO: 56 - protein, 62 aa, Human
EYHAEGGKVPIKWMALESILHRIYTHQSDVWSYGVTVWELMTFGSKPYDGIPASEISSILEK

SEQ ID NO: 57 - protein, 20 aa, Human
GERLPQPPICTIDVYMIMVK

SEQ ID NO: 58 - protein, 11 aa, Human
CWMIDADSRPK

SEQ ID NO: 59 - protein, 10 aa, Human
FRELIIEFSK

SEQ ID NO: 60 - protein, 91 aa, Human
  VARIANT 21,56: Phosphorylated amino acid
MARDPQRYLVIQGDERMHLPSPTDSNFYRALMDEEDMDDVVDADEYLIPQQGFFSSPSTSRTPLLSSLSATSNNSTVACIDRNGLQSCPIK

SEQ ID NO: 61 - protein, 38 aa, Human
EDSFLQRYSSDPTGALTEDSIDDTFLPVPEYINQSVPK

SEQ ID NO: 62 - protein, 61 aa, Human
RPAGSVQNPVYHNQPLNPAPSRDPHYQDPHSTAVGNPEYLNTVQPTCVNSTFDSPAHWAQK

SEQ ID NO: 63 - protein, 19 aa, Human
GSHQISLDNPDYQQDFFPK

SEQ ID NO: 64 - protein, 9 aa, Human
EAKPNGIFK

SEQ ID NO: 65 - protein, 22 aa, Human
GSTAENAEYLRVAPQSSEFIGA

SEQ ID NO: 66 - protein, 28 aa, Human
RPAGSVQNPVYHNQPLNPAPSRDPHYQD

(19) United States
(12) Patent Application Publication
(10) Pub. No.: US 2008/0280317 A1
(43) Pub. Date: Nov. 13, 2008

(54) Comprehensive Characterization Of Complex Proteins At Trace Levels

(75) Inventors: Shiaw-Lin Wu, Weston, MA (US); William S. Hancock, Brookline, MA (US); Barry L. Karger, Newton, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: NORTHEASTERN UNIVERSITY, Boston, MA (US)

(21) Appl. No.: 11/661,525

(22) PCT Filed: Aug. 29, 2005
(86) PCT No.: PCT/US2005/030713

§ 371 Date: Feb. 27, 2007

Related U.S. Application Data
(60) Provisional application No. 60/605,058, filed on Aug. 27, 2004.

(51) Int. Cl. C12Q 1/37; G01N 33/68; C12Q 1/00; B01D 57/02

(52) U.S. Cl. 435/23; 436/86; 435/4; 204/451

(57) ABSTRACT

A combination of “bottom up” and “top down” MS analysis of posttranslational modifications in complex proteins is described. The method comprises digestion of the protein with an enzyme that forms larger peptide fragments than trypsin (>3000 D), performing HPLC with the fragments and applying a new data acquisition strategy using on-line coupling with e.g. LTQ-FTMS, a hybrid mass spectrometer that couples a linear ion trap with a Fourier transform ion cyclotron resonance (FTICR) cell. The method is applied to analysis of posttranslational modifications of protein isoforms.

### CROSS REFERENCE TO RELATED APPLICATIONS

This application claims the priority of U.S. Provisional Application No. 60/605,058 filed Aug. 27, 2004 entitled, CHARACTERIZATION OF PROTEINS USING LARGE PEPTIDE FRAGMENT ANALYSIS, the whole of which is hereby incorporated by reference herein.

### STATEMENT REGARDING FEDERALLY SPONSORED RESEARCH OR DEVELOPMENT

Part of the work leading to this invention was carried out with United States Government support provided under a grant from the National Institutes of Health, Grant No. GM-15847. Therefore, the U.S. Government has certain rights in this invention.

### BACKGROUND OF THE INVENTION

The comprehensive characterization of proteins at trace levels in a biological sample is a significant challenge. Two strategies are currently widely available for protein analysis by mass spectrometry. The first, the bottom-up or shotgun approach,^{1,2} begins with the digestion of a protein (or proteome) with an enzyme, such as trypsin, followed by separation of the resulting peptides and analysis by mass spectrometry. A typical experimental design uses electrospray ionization with an ion trap mass spectrometer.^{1,3} However, many peptides are not detected either because they are too small (less than 500 Da) or too hydrophilic (multiple phosphorylation or glycosylation sites) to be well-retained on reversed phase LC columns. Conversely, large peptides are not recognized by most types of mass spectrometers because fragments over 5,000 Da require a resolution of 25,000 or higher to resolve the charge states and determine monoisotopic masses. Ion suppression can be a problem, and data-dependent analysis in on-line LC-MS often fails to detect co-eluting peptides.³ Because of these issues, the traditional bottom-up approach does not generally provide comprehensive characterization, due to limited sequence coverage and failure to identify posttranslational modifications.^{4,5}

The second strategy, the top-down approach, involves the analysis of intact proteins introduced by direct infusion into a Fourier transform mass spectrometer (FTMS).^{6,7} Using electrospray ionization, a number of multiply-charged ions of each protein are observed.⁸ Because these charge states are of a single protein, their m/z peaks can be mathematically deconvoluted to obtain the molecular weight. More recently, with high resolution, high mass-accuracy instruments such as FTMS, the charge can be directly determined from the ion envelope detected at high mass resolution, rather than by deconvolution.⁹ This approach is particularly useful when the species of interest are present in several isoforms, making determination of discrete charge states at low mass resolution highly problematic.⁹ Moreover, the high mass accuracy in FTMS matches fewer candidate peptide sequences and provides higher probabilities of correct assignments. The top-down approach has been used to obtain high sequence coverage of proteins as well as to determine alternative splicing or posttranslational modifications.¹⁰⁻¹⁴

However, the direct top-down approach (i.e., no enzymatic digestion) is generally applicable only for small to moderate size proteins (<50 kDa) with relatively homogeneous modifications.¹⁰ It is also generally difficult in the infusion of an intact protein to resolve the isotopic patterns of the multiple isoforms, each with a wide charge distribution, even when using the very high resolution of FTMS. Also, the top-down approach can require large amounts of material (often >10 pmole per protein),^{13,15,16} and the time required to isolate, fragment, detect and then remove the ions from the FTICR cell (>10 seconds per scan) is not compatible with on-line LC-MS. Moreover, the LC separation of intact protein mixtures, particularly with heterogeneous modifications, generally is much poorer than that of peptide mixtures.

Recently, a new on-line, top-down LC-MS strategy has been developed. This method uses a hybrid linear ion trap-FTMS instrument (LTQ-FTMS), which has been applied to the characterization of the disulfide linkages of intact human growth hormone, hGH (22 kDa), in a complex matrix of E. coli inclusion bodies, with detection at the 200 fmole level.¹⁷ In this method, the high mass resolution (typically 100,000) of the FTICR cell allowed the high charge states of intact human growth hormone precursor ions (15+ to 19+) and their large peptide fragments (10+, 11+) to be on-line determined with high mass accuracy (below 2 ppm).¹⁷ In this work, the LTQ-FTMS instrument was able to isolate in an on-line LC-MS experiment low abundance, highly charged (17+) ions of the intact protein in the FTICR cell, and to fragment these ions in the external linear ion trap cell using collisionally-induced dissociation (CID). Importantly, the fragment ions were transferred to and detected in the FTICR cell. The design of this hybrid instrument system permits such experiments to be performed on a chromatographic timescale.^{18,19}

In the above LC-MS top-down strategy,¹⁷ the MS and MS² spectra had to be measured in the FTICR cell in order to determine the high charge states associated with the high molecular weights of both the precursor and fragment ions. This is a drawback in an on-line LC-MS analysis, however, because the measurement in the FTICR cell requires 30- to 40-fold more time than measurement in the linear ion trap to achieve the same detection sensitivity. Furthermore, hGH, although relatively small, is about at the upper limit of proteins that can be analyzed accurately by this method. As noted above, the difficulty of resolving isotopic patterns of heterogeneous modifications increases with the size of the protein, becoming highly problematic above about 50 kDa. Thus, improved methods of characterizing proteins at trace levels continue to be desirable.

### BRIEF SUMMARY OF THE INVENTION

The invention is directed to a new and sensitive LC-MS platform, Extended Range Proteomic Analysis, which is able to achieve very high sequence coverage and comprehensive characterization of posttranslational modifications in complex proteins at the trace level (e.g., low pmole to fmole). The platform according to the invention provides advantages of both the top-down and bottom-up proteomic approaches by combining, In a preferred embodiment of the method, (i) digestion of the protein with an enzyme, such as Lys-C, that cuts less frequently than trypsin, or limited digestion with, e.g., trypsin, leading to, on average, a higher molecular weight peptide size with greater than 90% of the protein's peptide backbone sequence contained in fragments that are between 500 and 25,000 Da; (ii) high-performance LC separation of these resulting fragments; (iii) a new data acquisition strategy using on-line coupling of specific separated fragments to analysis in, e.g., the LTQ-FTMS, a hybrid mass spectrometer that couples a linear ion trap with a Fourier transform ion cyclotron resonance (FTICR) cell, for peptide analysis, preferably of the fragments in the range of 3000 to 10,000 Da; and (iv) new data analysis methods for assigning large peptide structures and determining the site of attachment of posttranslational modifications as well as structural features from the accurate precursor mass together with MS² and MS³ fragmentations.

The LC retention of the (e.g., Lys-C) fragments is increased, relative to a tryptic digest, due to the generally greater hydrophobicity of the larger peptides, a result that is particularly important for peptides containing hydrophilic modifications such as glycosylation and phosphorylation. Furthermore, additional positively charged arginine and lysine residues, which might be included in these larger fragments, could enhance the sensitivity of the posttranslationally modified peptides by at least 10-fold relative to tryptic fragments.

In a typical operation following production of these larger peptide fragments, the FTICR cell provides a survey scan with the high mass resolution (>100,000-200,000) and accurate mass (<2 ppm) needed to characterize the higher charge-state precursor ions of the larger peptides. In parallel, the linear ion trap provides MS² and MS³ fragmentation spectra, with a scan speed sufficiently fast for on-line LC-MS. Together, these data provide multiple means to determine or enhance the confidence of assignment of large or complicated peptides.

Using the method of the invention, we have demonstrated >95% sequence coverage in the analysis of the heavily phosphorylated protein, bovine beta-casein (at the 5-50 fmole level) and the heavily phosphorylated and glycosylated protein epidermal growth factor receptor (EGFR) (at the 20-200 fmole level). The detectibility range is also a function, e.g., of the size of the column used for fragment separation. This combination of digestion strategy, high-performance separation and use of the hybrid LTQ-FTMS instrument according to the method of the invention enables comprehensive characterization of large proteins, including their posttranslational modifications.

Thus, in general, the invention is directed to a method of protein characterization comprising providing an aliquot of a sample that includes a protein or a mixture of proteins whose identity is to be determined; carrying out digestion of the protein or mixture of proteins in the aliquot so that the digestion product comprises at least one fragment having a peptide backbone sequence of greater than or equal to 3000 (preferably, greater than or equal to 4000) Da in mass; separating the digestion product; and analyzing the structure of one or more of the fragments by mass spectrometry using a mass spectrometer system that includes a mass spectrometer having a mass resolution of at least 25,000 and a mass spectrometer having an electron multiplier detector. Preferably, the separating and analyzing steps are coupled on-line and the mass spectrometer system includes a mass spectrometer having a mass resolution of at least 50,000. The mass spectrometer system may include separate mass spectrometer instruments each having one of the indicated properties. Alternatively, the mass spectrometer system may include a single mass spectrometer instrument, such as a hybrid instrument.

The digestion step may be accomplished by proteolytic enzyme digestion, such as by one of the enzymes Lys-C, Arg-C and Asp-N, which tend to product larger fragments, or by limited digestion with a more frequent cutter, such as trypsin or Glu-C, to also produce larger fragments. Alternatively, digestion may be by a chemical reaction, with such compounds as dilute acid, cyanogen bromide and hydroxylamine. In a related aspect, in the digestion product, greater than 90% of the peptide backbone sequence of the protein or proteins is contained in fragments that are between 500 and 25,000 Da in mass, or more preferably between 1000 and 10,000 Da in mass. In particular embodiments, the digested fragments are separated using liquid chromatography or capillary electrophoresis, e.g., on a microfluidic chip. Alternatively, capillary electrochromatography is the method of choice.

Preferably, the mass spectrometer having an electron multiplier detector is an ion trap or quadrupole mass spectrometer and the mass spectrometer with a mass resolution of at least 25,000 is a Fourier transform mass spectrometer, a time-of-flight mass spectrometer or an Orbitrap. More preferably, the mass spectrometer system is a hybrid mass spectrometer that couples an ion trap with a Fourier transform ion cyclotron resonance cell is used. Alternatively, the mass spectrometer system is a hybrid mass spectrometer that couples a quadrupole mass spectrometer with a time-of-flight mass spectrometer is used. Most preferably, the detectors of the hybrid mass spectrometer are operated in parallel.

In another aspect, the method of the invention is carried out to determine the identity of different posttranslationally modified isoforms of a protein. For this use of the method, the sample includes a protein or mixture of proteins whose posttranslationally modified isoforms are to be determined. Following the digestion and separation steps, the position of fragments containing the common backbone of the posttranslationally modified isoforms is determined and the structure of the fragments containing the common backbone of the posttranslationally modified isoforms is analyzed according to the method of the invention. Preferably, the level of the individualized posttranslationally modified isoforms is quantitatively determined. In one embodiment, the posttranslational modification includes glycosylation, in another the posttranslational modification includes phosphorylation. other possible posttranslational modifications include sulfation, acetylation or methylation. In a further embodiment, the glycosylated posttranslational modification is further modified with sulfation and/or phosphorylation.

The examples described herein are provided to illustrate advantages of the invention, including those that have not been previously described, and to further assist a person of ordinary skill in the art with using the methods of the invention. The examples can include or incorporate any of the variations or inventive embodiments as described herein. The embodiments that are described herein also can each include or incorporate the variations of any or all other embodiments of the invention. The following examples are not intended in any way to otherwise limit or otherwise narrow the scope of the disclosure as provided herein.

### BRIEF DESCRIPTION OF THE SEVERAL VIEWS OF THE DRAWINGS

Other features and advantages of the invention will be apparent from the following description of the preferred embodiments thereof and from the list of embodiments, taken in conjunction with the accompanying drawings, in which:

FIG. 1 shows a data acquisition scheme using a hybrid linear ion trap-Fourier transform mass spectrometer (LTQ-FTMS) according to the method of the invention;

FIG. 2 shows the amino acid sequence of bovine beta-casein (209 amino acids, molecular weight 23,568 Da) and the peptide sequences resulting from a theoretical Lys-C digest. Additional trypsin cleavage sites are indicated by arrows at the R residues. There is one likely missed cleavage between two adjacent lysines at positions 28 and 29. The five known phosphorylation sites are indicated as S*, at residues 15, 17, 18, 19, and 35. The two peptides with molecular weight greater than 5,000 Da are underlined (residues 49-97, and 114-169). The three peptides with molecular weight less than 500 Da are too short to be analyzed (residues 30-32, 98-99, and 106-107);

FIG. 3 shows analysis of a long peptide fragment from the Lys-C digestion of beta-casein, using the hybrid LTQ-FTMS instrument according to the method of the invention. Panel A: total ion chromatogram. Panel B: precursor mass scan at 35.01 min using the FTICR (single scan from 400 to 2000 m/z with 100,000 resolution and 2×10⁶ target ions). For illustration purposes, only the 1271 to 1276 m/z mass region of 5+ ions are shown. The data-dependent acquisition mode was used to isolate, with a ±2.5 m/z isolation width, ions with the sequentially highest intensities from the MS list for MS² fragmentation in the linear ion trap. Panel C: MS² scan of the m/z 1273.2598 ion (single scan from m/z 343 to 2000). The data-dependent acquisition mode was again used to isolate automatically the highest-intensity MS² ion for MS³ fragmentation. Panel D: MS³ scan of the m/z 1237.1 ion above (single scan from m/z 334 to 2000). The peptide sequence identified by Sequest from the MS² spectrum is shown in the insert of panel B, with the sequence from the MS³ spectrum underlined;

FIG. 4 shows analysis of the tetraphosphorylated fragment from the Lys-C digestion of beta-casein, using the hybrid LTQ-FTMS instrument according to the method of the invention. Panel A: total ion chromatogram. Panel B: precursor mass scan at 26.32 min using the FTICR. For illustration purposes, only the m/z 1158-1163 region is shown. Panel C: MS² scan of the m/z 1160.1696 ion. Panel D: MS³ scan of the m/z 1127.7 ion. The peptide sequences identified by Sequest are shown in the inserts. The phosphorylation site is indicated as S*, the neutral loss of phosphate site as S#, and the tryptic cleavage sites (R or K) are highlighted in bold. Mass spectrometer conditions are as described for FIG. 3;

FIG. 5 shows analysis of a long peptide fragment from the Lys-C digestion of EGFR, using the hybrid LTQ-FTMS instrument according to the method of the invention. Panel A: total ion chromatogram. Panel B: precursor mass scan at 29.27 min using the FTICR (only the 1130 to 1137 m/z mass region of 6+ is shown). Panel C: MS² spectrum of the m/z 1133.0632 ion. Panel D: MS³ scan of the m/z 1046.4 ion. The peptide sequences identified by Sequest are shown in the inserts. Mass spectrometer conditions are as described for FIG. 3;

FIG. 6 shows analysis of a large phosphorylated peptide fragment from the Lys-C digestion of EGFR, using the hybrid LTQ-FTMS instrument according to the method of the invention. Panel A: total ion chromatogram. Panel B: precursor mass scan at 35.82 min using the FTICR (only the m/z 758 to 760 mass of 5+ is shown). Panel C: MS² scan of the m/z 759.2357 ion. Panel D: MS³ scan of the m/z 739.7 ion above. The peptide sequences identified by Sequest are shown in the insert; the phosphothreonine is indicated as T*, the neutral loss of phosphate site as T#. Mass spectrometer conditions are as described for FIG. 3;

FIG. 7 shows analysis of a glycosylated peptide fragment, modified with a high-mannose-type glycan, from the Lys-C digestion of EGFR, using the hybrid LTQ-FTMS instrument according to the method of the invention. Panel A: total ion chromatogram. Panel B: precursor mass scan at 36.15 min using the FTICR (only the 1423 to 1433 m/z mass region of 4+ is shown). Panel C: MS² scan of the m/z 1427.4321 ion. Panel D: MS³ scan of the m/z 1403.5 ion above. The peptide sequences shown in the insert of Panel B were identified manually, as described herein. The glycosylation site is labeled N*, and the tryptic cleavage sites (R or K) are highlighted in bold. In the Man8 structure, triangle (▴) represents mannose and circle () represents N-acetyl glucosamine. The sequential losses of terminal mannoses from the Man8 structure resulted in Man7, Man 6, etc., as indicated in Panel C. Potential antenna structures are also indicated. Mass spectrometer conditions are as described for FIG. 3; and

FIG. 8 shows analysis of a peptide fragment modified with a complex-type glycan, from the Lys-C digestion of EGFR using the hybrid LTQ-FTMS instrument according to the method of the invention. Panel A: total ion chromatogram. Panel B: precursor mass scan at 36.70 min using the FTICR (only the 1167 to 1173 m/z mass region of 4+ is shown). Panel C: MS² scan of the m/z 1169.3159 ion. Panel D: MS³ scan of the m/z 1340.4 ion above. The peptide sequences shown in the insert of Panel B were identified manually, as described herein. The glycosylation site is labeled N*, and the tryptic cleavage sites (R or K) are highlighted in bold. In the glycan structures, SA represents sialic acid, square (▪) represents galactose, triangle (A) represents mannose and circle () represents N-acetyl glucosamine. Additional glycan cleavage fragments, such as antennae, are also indicated. Mass spectrometer conditions are as described for FIG. 3.

### DETAILED DESCRIPTION OF THE INVENTION

To overcome the limitations on protein size and heterogeneity and the long detection times required to achieve FTICR sensitivity in the top-down approach, we have developed a new intermediate and sensitive strategy called Extended Range Proteomic Analysis, a method that combines key features of the top-down and bottom-up approaches along with more productive use of the LTQ-FTMS instrument.

This new platform, for the first time, allows for the characterization of the complete structure of a protein present in a complex biological mixture. In the past, such analyses were only possible, in a limited sense, for a protein that had been extensively purified and was available in substantial amounts. Even in that situation, such an analysis was problematic in that one would not know if a particular set of modifications were indeed present in a given species. For example, if one characterizes a specific phosphorelation in a peptide and then in a separate analysis characterizes a sulfation in a carbohydrate site chain in another reasonable molecule (as found in another peptide fragment) there is no guarantee that both modifications are present in the same protein species. In fact, the situation may well be that the two modifications reside in separate protein molecules as a consequence of the biological heterogeneity of protein biosynthesis.

This lack of ability to characterize the structure of a protein completely, and in trace amounts, has important consequences in the biotechnology industry. Currently, the FDA regulates a protein pharmaceutical by both final product testing and characterization of the production process. Although protein products can be termed as being adequately characterized, the current state of technology does not allow for the production of “a well characterized biological.” Such a situation has clear ramifications when a pharmaceutical is produced in different countries with materials from different suppliers or when one wishes to produce a generic biotechnology drug. In a similar manner, the lack of complete characterization of proteins has major implications in the pharmaceutical industry both in terms of characterizing drug targets and in development of small molecule pharmaceuticals based on inhibition of specific protein interactions. One can also see that the monitoring of patients for clinical trials is hindered by this lack of knowledge as well.

The problem is that current technology has been unable to characterize large peptide fragments, particularly in proteins available in small amounts, such as those present in complex biological samples. The major thrust of this invention, then, is a technology that allows the characterization of overlapping peptide fragments and includes a process by which a nested set of fragments can be put together to describe the complete protein sequence with detailed knowledge of the occurrence of post-translational modifications.

In the method according to the invention, a protein to be analyzed is cleaved, preferably, using an enzyme that cuts less frequently than trypsin (or by incomplete digestion. e.g., with trypsin) to obtain a smaller number of peptides, many of which are of higher molecular weight than the tryptic fragments produced in the traditional bottom-up approach. Thus, the problems of the heterogeneity of intact proteins as well as the complexity of tryptic peptides are both decreased. Another important point is that trypsin will generate peptides that carry the positively-charged K or R residues only at their C-terminus, while peptides generated by, e.g., Lys-C digestion frequently include one or more additional R residues in their internal sequence. As will be shown, when glycosylated or phosphorylated peptides carry this additional positive charge, their MS signal intensity is enhanced 10-fold or higher and additional b ions are found in the fragmentation spectrum. In the method of the invention, the FTICR cell is used to resolve the higher charge states and obtain accurate mass measurements of the larger peptides (2- to 3-fold larger than typical tryptic peptides), while concurrently taking advantage of the speed and sensitivity of the linear ion trap for CID measurements on these peptides. In addition, because current software (e.g., Sequest, Mascot) does not address significance thresholds for peptides above a 3+ charge state, and because only limited software exists for automatically recognizing glycopeptides or assigning glycans, the method of the invention includes new strategies for these tasks.

### Uses

The method of the invention will be very useful in the pharmaceutical and biotechnology industries, e.g., for applications such as the following: characterization of a protein product in a fermenter; tracking of even trace amounts of impurities during the purification process; final product testing and characterization for an FDA submission; improved QC testing using peptide maps (fewer fragments give a less complicated map with better coverage of the N and C-terminus of the protein).

These applications will be especially valuable for antibody drugs. For example, a fermentation sampler can be coupled to an antibody capture column that will extract the product away from the other fermentation proteins. The captured protein can then be released, and digested and analyzed according to the method of the invention.

Earlier in the drug development process, the method will find use for, e.g., drug target characterization, especially in the definition of the full sequence of a target including combinations of post translational modifications (PTMs). Specifically, cell line extracts can be analyzed to look at the effect of drugs on specific protein targets. Another use can be for signalling pathway definition, e.g., by providing for a detailed analysis of the level of phosphorylation of a receptor on the inner surface of a plasma membrane and of glycosylation motifs on the outer surface.

In general, in the method of the invention, the posttranslationally modified fragments produced are sufficiently large, and, thus, have a sufficiently large signal, that all isoforms of a modification at a specific site will elute at the same position. Thus, a quantitative determination can be made of the individualized isoforms. Examplary modifications include glycosylation, phosphorylation, sulfation, acetylation, methylation and other forms suitable for a specific purpose, such as a general membrane anchor. The modified groups attached to the peptide backbone of the protein have been observed to themselves be modified.

The method will also allow for the characterization of large peptide fragments without any enzyme digestion. This is an invaluable resource for the study of the peptidome or fragmentome. This application would involve a MW separation step (e.g., gel permeation chromatography or a membrane filter step) and characterization of the low MW fraction.

The use of the method of the invention for biomarker discovery in complex fluids such as plasma, where low level proteins are often identified with a single peptide, will be very helpful. With the large fragment analysis approach of the invention, a sample can be split into several (typically three) aliquots and digested with enzymes that produce large fragments. The separate aliquots can be analyzed individually according to the method of the invention and the peptide identifications pooled. In this way also, the forms, e.g., glycosylated forms, of the biomarker in a specific patient can be monitored, for individualized treatment with the appropriate therapy.

The method of the invention would also find application in the discovery of interacting proteins. This application would involve an affinity system (e.g., magnetic particles or binding agent such as an antibody on an affinity column) to isolate the target protein, e.g., EGFR, complexed with a ligand. The recovered complex can then be crosslinked with a crosslinking agent, such as glutaraldhyde. The crosslinked sample is then digested, and the fragments are characterized using the method of the invention, which will give the region of the structure of the target protein and the corresponding region of the interacting protein.

Using the method of the invention, improved protocols can be developed for MS identification of protein mixtures and proteomic samples. For example, this approach can be used to reduce the rate of false positives and can be combined with genomic databases to allow for unique identification of a protein member of a family. The method of the invention will also support the development of improved methods of determining missing regions in a protein as a result of proteolysis and/or alternative splicing.

In summary, by providing the ability to characterize large overlapping fragments, the method of the invention allows for the first time the determination of the full structure of a protein with very high sequence coverage and comprehensive characterization of posttranslational modifications, even at the trace level. High sequence coverage is particularly important for determining the tissue or sub-cellular compartment of origin of a specific posttranslationally modified variant.

The following examples are presented to illustrate the advantages of the present invention and to assist one of ordinary skill in making and using the same. These examples are not intended in any way otherwise to limit the scope of the disclosure.

To demonstrate the power of this method, the examination of two proteins with extensive posttranslational modifications is described herein: phosphorylated beta-casein (23 kDa), and the glycosylated and phosphorylated epidermal growth factor receptor (EGFR) (180 kDa). The advantages of the method according to the invention are shown in a comprehensive characterization of these proteins, including the identity and attachment sites for all significant phosphorylated and glycosylated peptides, with high sequence coverage (>95%) and a high sensitivity for beta-casein and EGFR. The approach will be directly applicable to the comprehensive analysis of protein biomarkers or protein complexes that have been isolated from biological matrices, for example, by immunoprecipitation.

As discussed above, the two most common mass spectrometric approaches for the characterization of proteins are direct analysis of intact proteins (top-down), or analysis of a mixture of peptides resulting from a tryptic digest (bottom-up). In the top-down approach, if the intact protein is larger than 50 kDa or has heterogeneous modifications, comprehensive analysis is highly challenging if not impossible. The bottom-up approach has low detection sensitivity for glycosylated or phosphorylated peptides in a mixture of non-modified tryptic peptides.^{21,22} The method of the invention, Extended Range Proteomic Analysis, described herein is an alternative and sensitive approach. Given below are, first, important general considerations in this approach and, then, analysis of several complex proteins as examples of using this method.

### Choice of Proteolytic Enzyme

Trypsin is the most commonly used proteolytic enzyme today, in part because it is “well behaved”²³ and in part because the majority of fragments tend to be below 3 kDa, which is compatible with present day ESI-MS instruments. However, with the new generation of instruments, such as the LTQ-FTMS, larger fragments with higher charge states can be routinely analyzed. This opens up the possibility of directly using enzymes other than trypsin that cut proteins less frequently.

We examined the theoretical digest of the human SwissProt database using 3 different enzymes (Glu-C, trypsin and Lys-C) and one chemical reagent (cyanogen bromide). The majority of the resulting digest fragments were below 3 kDa for Glu-C and trypsin, 3 to 10 kDa for Lys-C, and 5 to 20 kDa for cyanogen bromide. In terms of the number of peptides, Glu-C and trypsin generated 2- to 3-fold more peptides than Lys-C, and 5-fold more than cyanogen bromide. Because Lys-C created many peptides in the desired 3-10 kDa size range, it was selected for initial practice of the method of the invention. Further examination of the human proteome revealed that the occurrence of R and K residues in proteins was roughly equivalent at 6%. From these percentages, it can be predicted that most Lys-C peptides will have at least one additional R or K residue, relative to the corresponding tryptic peptides. As shown in the following results, this positive charge can improve the electrospray ionization efficiency at least 10-fold for posttranslationally modified peptides carrying the negative charge of phosphorylation or sialylation, when operating under the typical conditions of positive ion mode and low pH buffers. In addition, Lys-C is a robust enzyme which can digest proteins even under harsh conditions, such as 6 M urea or 0.5% SDS. As discussed below, we also explored the strategy of further digesting a portion of the Lys-C digest with trypsin, as necessary, if a Lys-C fragment was much larger than 10 kDa.

### Operation of the Linear Ion Trap-FTMS

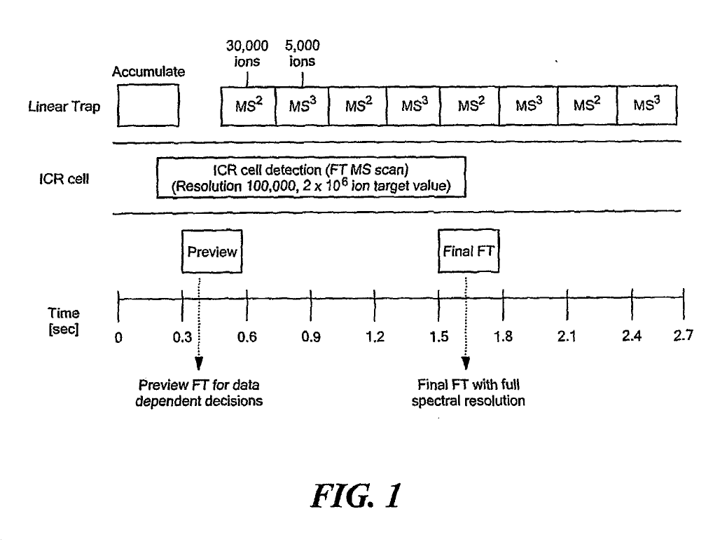

As shown in FIG. 1, the LTQ-FTMS is a hybrid instrument with two independent detectors, the FTICR and a linear ion trap, both of which can be operated in parallel. The acquisition time for the FTICR cell is proportional to both the ion target value and mass resolution of the scan. Higher ion target values provide better ion detection, at the price of longer times to fill the cell and the introduction of space-charge effects (which degrade mass accuracy). We chose an ion target value of 2×10⁶, which would provide a mass accuracy of approximately 2 ppm and an FTICR sensitivity comparable to the linear ion trap loaded with 30,000 ions. Similarly, higher mass resolution requires a longer FTMS scan time but is necessary when peptides co-elute or are highly charged. With a resolution of 100,000 and an ion target value of 2×10⁶, the FTICR scan time is 1.8 seconds. The linear ion trap in the LTQ-FTMS has a 10-fold higher ion storage capacity than standard 3D ion traps.²⁴ With this high trapping capacity and a fast scan speed, fragmentation scans in the linear ion trap are approximately 0.3 second, depending on the number of microscans used and the abundance of ions eluting at a given LC time point.

The FTICR preview was followed by 8 MSⁿ scans (typically 4 pairs of linear ion trap MS² and MS³ scans). The total cycle time was 2.7 seconds. The acquisition time for the full FTICR scan required 1.8 seconds with an ion target value of 2×10⁶ and a resolution of 100,000, as in the scheme above. Five MSⁿ linear ion trap scans could be acquired concurrently within this 1.8 sec (parallel acquisition), with each additional MSⁿ scans adding 0.3 sec to the cycle time. Each MS² CID fragmentation (target value of 30,000 ions) was performed on a precursor ion that was isolated using the data-dependent acquisition mode to select ions automatically in sequence from the highest intensities of the preview scan. From the MS² fragmentation spectrum, a subsequent MS³ CID fragmentation (at a target value of 5,000 ions) was performed on a precursor ion from the MS² scan, isolated using the data-dependent acquisition mode to select automatically ions with the highest intensity.

FIG. 1 shows that, with each detector operating in parallel, the linear ion trap can acquire roughly 5 MSⁿ spectra during the 1.8 sec that the FTICR requires to determine a single high-resolution MS spectrum. The MSⁿ spectra include MS² plus additional MS³ or higher stages to enhance the confidence of the peptide assignments.²⁵ Additional (more than 5) MSⁿ scans each add about 0.3 sec to the cycle time during an on-line LC-MS analysis; however, the FTICR scan time is still 1.8 seconds. In our experience, a total cycle time of approximately 3 seconds provides a good balance for effective on-line LC-MS analysis, acquiring 1 FTICR scan plus 8 MSⁿ linear ion trap scans (4 pairs of MS² and MS³ scans in the linear ion trap) in each cycle (see FIG. 1). Typically, the MS³ fragmentation is conducted on the highest intensity fragment ion from the prior MS² spectrum.

### Identification of Large Peptides

Central to the strategy in the method of the invention is the ability to identify, with high confidence, peptides that can be 2- to 3-fold greater in length than normally produced in tryptic digests. Current database search software (e.g., Mascot and Sequest) has been optimized for use with tryptic peptides (<3,000 Da) and, although the current version of Sequest will process ions with charge states up to 8+, no statistics are available correlating the probability of correct identification to the X_{corr} scores of ions above the 3+ charge state. In the absence of this information, our present strategy for analysis of large peptides with charge states of 4+ or higher is to use the current version of Sequest (BioWorks 3.1 SR1) as a means to select and rank the most likely candidates, then to confirm the sequences manually using (i) accurate mass measurement (within 2 ppm) provided by the FTMS, (ii) agreement between the MS² and MS³ identifications, and (iii) expected cleavage ions in the CID spectra. As the database search software improves, less manual confirmation will be needed.

Interestingly, we have found that CID fragmentation of large precursor ions with higher charge states leads to a low number of high-intensity preferred-cleavage fragments rather than a complete ion series. Also, although X_{corr} thresholds of significance increase with charge states from 1+ to 3+ (e.g. >1.9 for 1+ ions, >2.2 for 2+ ions, and >3.75 for 3+ ions), our preliminary findings suggest that X_{corr} threshold scores of ions of 4+ or higher charge are similar to those for 3+ ions. This trend could reflect the fact that larger precursor ions have fewer potential peptide candidates in the database, especially when using strict precursor ion mass tolerance and proteolytic enzyme specificity. For example, searching against the SwissProt database using Lys-C specificity with zero missed cleavages and ±10 ppm mass accuracy, we found roughly 60 potential peptide candidates at MH+=2,000 Da, as compared to only 5 candidates at MH+=8,000 Da. Thus, with high mass accuracy FTMS data, the assignment of spectra of large peptides can be based on fewer fragment ion assignments than are required for small peptides. The analysis of two commercially available complex proteins, beta-casein and epidermal growth factor receptor, is given below to demonstrate the principles and effective results of this method.

### Beta-Casein

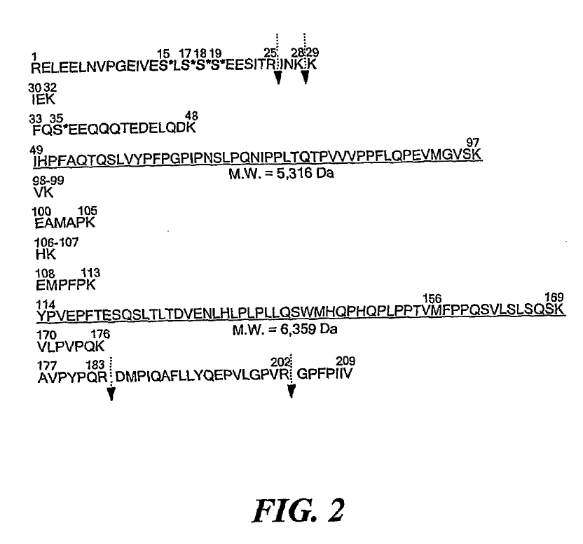

Bovine beta-casein, a 23 kDa protein containing 5 known serine phosphorylation sites, was chosen as a model phosphoprotein. ²⁰, ²⁶ Carrying out the method of the invention by conducting LC-MS on Lys-C fragments of the protein provided 97% sequence coverage (202 out of 209 amino acid residues), including the identification of all phosphorylation sites, at the 50 fmole level (see Table 1 and FIG. 2). In the following discussion, the key points of this analysis are illustrated. Because the identification of unmodified peptides smaller than roughly 3500 Da was similar to the traditional methods, we will discuss here only the analysis of the large and multiply-phosphorylated peptides.

TABLE 1
Peptides identified according to the invention^{a} - Bovine Beta-Casein

| | | | [M + H]⁺ | | |
|---|---|---|---|---|---|
| start | end | Peptide Sequence^{b} | (Da)^{c} | Identification^{d} | Charge States |
| 1 | 28 | RELEELNVPGEIVES*LS*S*S*EES | 3477.4879 | + | 3+, 4+ |
| | | ITRINK | | | |
| 1 | 29 | RELEELNVPGEIVES*LS*S*S*EES | 3605.5825 | + | 3+, 4+ |
| | | ITRINKK | | | |
| 30 | 32 | IEK | 389.2395 | − | − |
| 33 | 48 | FQS*EEQQQTEDELQDK | 2061.8284 | +^{e} | 2+, 3+ |
| 49 | 97 | IHPFAQTQSLVYPFPGPIPN | 5316.8533 | + | 4+, 5+, 6+, |
| | | SLPQNIPPLTQTPVVVPPFLQPEVMG | | | 7+ |
| | | VSK | | | |
| 98 | 99 | VK | 246.1812 | − | − |
| 100 | 105 | EAMAPK | 646.3228 | + | 1+ |
| 106 | 107 | HK | 284.1717 | − | − |
| 108 | 113 | EMPFPK | 748.3698 | + | 1+ |
| 114 | 169 | YPVEPFTESQSLTLTDVENLHLPLPL | 6359.2559 | + | 5+, 6+, 7+ |
| | | LQSWMHQPHQPLPPTVMFPPQSVLSL | | | |
| | | SQSK | | | |
| 170 | 176 | VLPVPQK | 780.4978 | + | 1+ |
| 177 | 209 | AVPYPQRDMPIQAFLLYQEPVLGPVR | 3721.0337 | + | 2+, 3+, 4+ |
| | | GPFPIIV | | | |

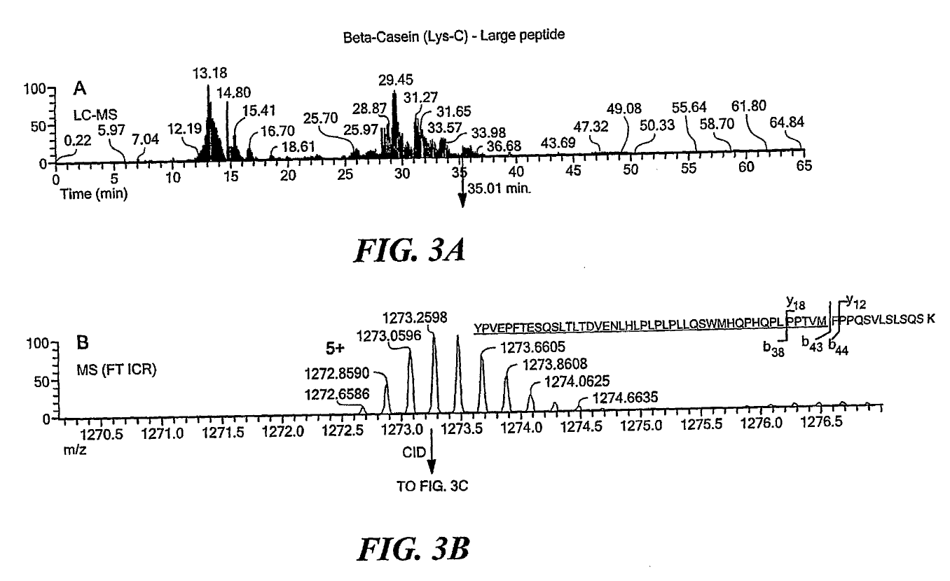

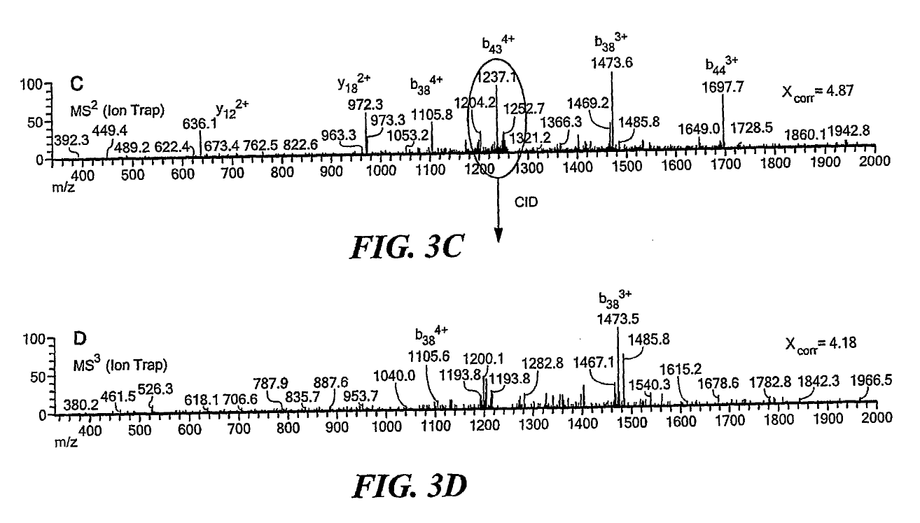

^{a}See FIG. 3 caption and Experimental Section for details.
^{b}“*” indicates phosphorylated amino acids (S*).
^{c}Theoretical monoisotope with a hydrogen mass [M + H]⁺ shown, including the molecular weight of phosphorylation if indicated.
^{d}“+” = identified, “−” = not identified.
^{e}Both phosphorylated and nonphosphorylated peptides found.

Referring to FIG. 2, it can be seen that a theoretical digest of bovine beta-casein using Lys-C yielded 11 peptide fragments, with three fragments being less than 500 Da (three amino acid residues or less) and four fragments being greater than 3500 Da, including two greater than 5000 Da. This calculation assumed no missed cleavages except: R or K residues on the C-terminal side of a P residue, two R or K residues adjacent to each other (RR or KK), or R or K residues at the N-terminus of the protein.²³ Referring again to FIG. 2, for comparison, a theoretical digest of trypsin yielded 14 peptides, of which four were less than 500 Da and two peptides were greater than 5,000 Da, the latter being identical to the Lys-C digest. As a result, three fewer peptides (11 vs. 14 peptides) and one less small peptide (<500 Da) would be generated using Lys-C instead of trypsin.

Experimentally, after digestion with Lys-C, beta-casein (50 fmole) was analyzed online by the method of the invention following LC separation over a 75 μm i.d. C-18 reversed phase column (300 Å pore). The total ion chromatogram is shown in FIG. 3, Panel A. At the indicated elution time of 35.01 min, a precursor ion with a 5+ charge state and an accurate monoisotopic mass of m/z of 1272.6756 (6359.2642 Da) was observed in the FTICR survey scan, shown in Panel B. The m/z 1272.7 ion was isolated by the data-dependent acquisition mode, which automatically selected the most intense ions from the survey scan (±2.5 m/z width), and subjected them to MS² CID fragmentation in the linear ion trap (Panel C). From this MS² spectrum, the m/z 1237.1 precursor ion was automatically selected for MS³ CID fragmentation (Panel D) based on its highest intensity. As described above, no additional time was introduced between the scans in the FTICR and the linear ion trap since both the FT survey scan and the ion-trap fragmentation scans occurred concurrently within the same chromatographic time window (35.01 to 35.06 min, in this case). From the MS² spectrum, Sequest identified the most likely peptide as beta-casein residues 114-169 (shown in the sequences of FIG. 2 and the insert in FIG. 3B) with an X_{corr} score of 4.87. An independent Sequest search of the MS³ spectrum identified the most likely peptide sequence of this daughter fragment as beta-casein residues 114-156, a partial sequence of the MS² assignment (underlined in FIG. 3B), with an X_{corr} of 4.18.

The MS² and MS³ database search results were then confirmed manually. First and foremost, the accurate mass measurement of the precursor ion (6359.264 Da) matched within 2 ppm the molecular weight of the peptide identified in the MS² assignment (6359.255 Da). Additionally, we compared the masses of the high intensity ions observed in the fragmentation spectra with those that would be generated by cleavage at preferential sites of the candidate sequence, specifically: cleavage between L and P (y18 and b38), between M and F (b43), and between F and P (y12 and b44), as indicated in the insert to Panel B and the MS² spectrum in Panel C of FIG. 3. When the b43 ion in the MS² spectrum was fragmented further, the b38 ion was the most intense peak in the MS³ spectrum (Panel D, FIG. 3). The combination of the MS² and MS³ assignments, the close matching of the theoretical and observed masses, and the consistency of the expected fragmentation sequences with the observed fragmentation, taken together, provide compelling evidence for the assignment of the 5+ charge state ion as the 6359 Da peptide, residues 114-169.

Another large peptide with a molecular weight of 5316 Da (residues 49-97 in FIG. 2), eluting at 33.37 min, was similarly assigned by the method of the invention (See Table 1). This large peptide, and that in FIG. 3, are common to both Lys-C and trypsin digests and together constitute 50% of the beta-casein sequence (105 out of 209 amino acid residues). Importantly, these peptides would have not been easily detected in the conventional shotgun approach using an ion trap mass spectrometer.¹

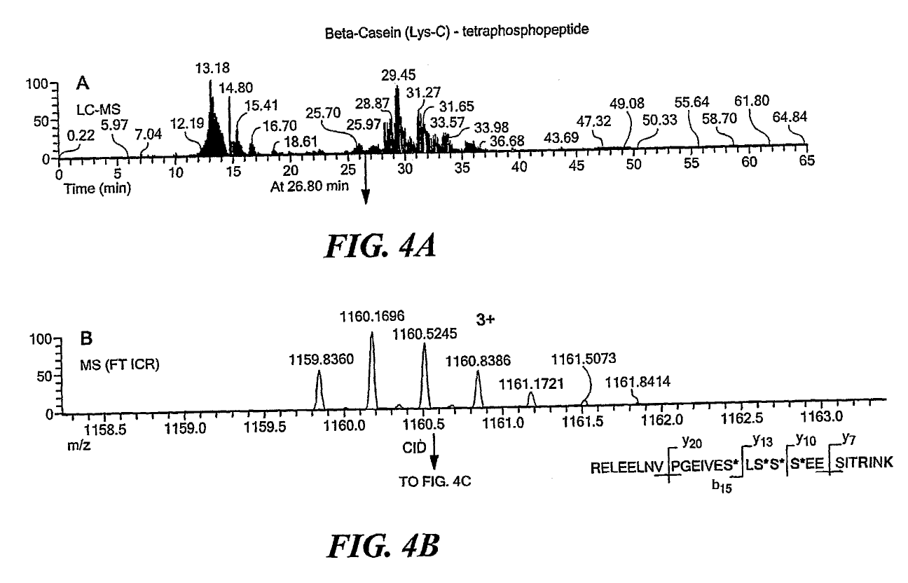

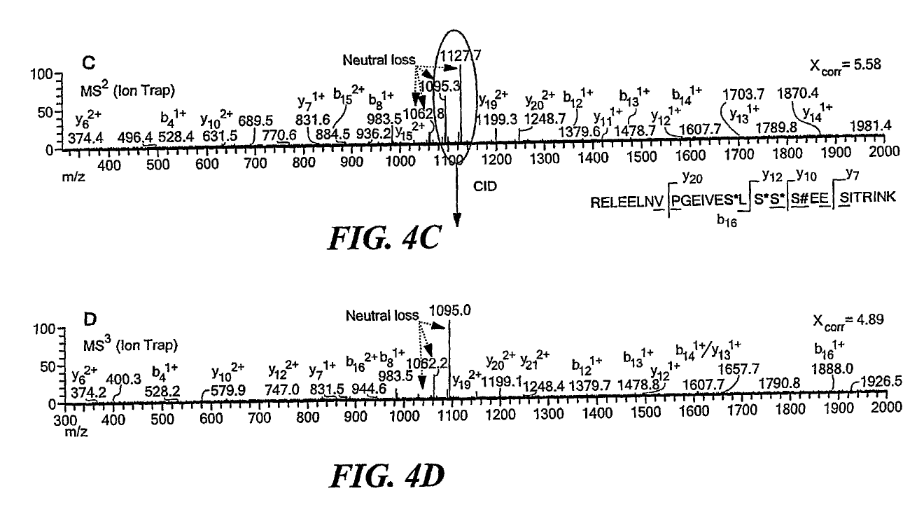

We next turn to examination of the tetraphosphorylated peptide. At an elution time of 26.80 min (FIG. 4, Panel A), a precursor ion was observed in the FTICR survey scan (Panel B) with a 3+ charge state and a monoisotopic mass m/z of 1159.8360 (3477.4936 Da). This ion was automatically isolated in the linear ion trap and fragmented by CID to produce the MS² spectrum (Panel C), from which the most intense ion (m/z=1127.7) was again automatically selected and fragmented to yield an MS³ spectrum (Panel D). The MS² fragmentation resulted in a ladder of 4 neutral loss ions (Δm=98, 196, 294, and 392 Da), indicative of multiple phosphorylation sites on the peptide. Sequest assigned this spectrum to residues 1-28 of beta-casein, shown in the inserts of Panels C and D.

As in FIG. 3, the accurate mass of the observed ion agreed with the predicted mass of the assigned sequence within 2 ppm. In addition, in the MS² spectrum (Panel C), the y20 ion (cleavage between V and P, underlined) was observed as a tetraphosphopeptide, whereas the y7 ion (cleavage between E and S, underlined) was seen as an unmodified peptide. Thus, the four phosphorylation sites were deduced to lie between the y7 and y20 fragmentation bonds, shown as S*. The y13, y10, and b15 ions further confirmed the assignment of the four phosphorylation sites (Panel C insert, FIG. 4). In the MS³ spectrum (Panel D), the peptide showed a 98 Da neutral loss at one S* site (y10 position) with phosphates remaining on the other three S residues (Panel D, S#=the neutral loss). This conclusion was clearly established through the assignment of the y20 ion (cleavage between V and P, underlined) and y10 ion (cleavage between S* and S#, underlined). Again, additional ions (y17, y12, and b16) further confirmed the sites of phosphorylation and neutral loss. Identifying the same phosphorylation sites in the MS³ spectrum reinforced the MS² assignments.

An important finding from the results shown in FIG. 4 was that the sensitivity of MS detection was greatly improved for the tetraphosphorylated peptide in the Lys-C digest, relative to the tryptic peptide (resulting from trypsin digestion following Lys-C digestion). With 50 fmole of beta-casein, we could not observe even the precursor ion of the corresponding tetraphosphorylated tryptic peptide RELEELNVPGEIVES*LS*S*S*EESITR, which had three fewer amino acids (INK) at its C-terminus than the corresponding Lys-C fragment. To observe the tryptic peptide, 1 pmole or more of the digest had to be injected. Given that there is little difference in molecular weight between the two peptide fragments, the additional K residue in the Lys-C fragment was likely the reason for the 20-fold difference in the limit of detection. The additional positive charge would aid in partially neutralizing the negative charge of the phosphate groups.

Finally, the fifth phosphorylation site for beta-casein was seen in the peptide sequence FQS*EEQQQTEDELQDK, eluting at 15.41 min. This site was readily identified with the same neutral loss analysis, and both the non-phosphorylated as well as phosphorylated forms of this peptide were observed (Table 1). Both trypsin and Lys-C digestion produced this same monophosphopeptide fragment.

Thus, 97% sequence coverage of beta-casein (202 out of 209 amino acid residues) was obtained using the approach according to the method of the invention at the 50 fmole level, including identification of all five phosphorylation sites. The only missing residues were the three di- and tri-peptides (IEK, VK, and HK), as shown in Table 1. In contrast, the typical tryptic bottom-up approach with the same quantity of protein would not have detected the tetraphosphorylated peptide nor the two large peptides in beta-casein, resulting in a sequence coverage of 36% at best.

### Human Epidermal Growth Factor Receptor (EGFR)

We now turn to the ability of the method of the invention to comprehensively characterize a large protein with heterogeneous phosphorylation and glycosylation structures. EGFR is a transmembrane glycoprotein comprising 1186 amino acids with a molecular weight of 132 kDa, based on the amino acid sequence. In SDS-PAGE, however, the receptor migrates with an apparent molecular weight of 180 kDa, which suggests the presence of posttranslational modifications, particularly glycosylation.²⁷⁻²⁹ EGFR is composed of three domains; an extracellular ligand-binding domain (residues 1-621), a transmembrane region (residues 622-644) and an intracellular cytoplasmic domain (residues 645-1186).³⁰ When activated, the cytoplasmic kinase domain of the receptor triggers signaling cascades within the cell that are implicated in a number of diseases.^{31,32} EGFR overexpression is a well-known biomarker in several cancers.³³ The capability to analyze such receptors comprehensively, including the posttranslational modifications which are indicative of their activation states, could thus offer important insights into a number of disease processes.^{34,35} Multiple phosphorylation sites have been reported for EGFR,^{29,36-38} and there are 12 theoretically possible N-linked glycosylation sites.^{30,39} Because of the large size and heterogeneous modifications of this protein, direct top-down analysis would be difficult and insensitive if it could be carried out at all.⁴⁰

A theoretical digest of EGFR using Lys-C yielded 63 peptide fragments, with 12 peptides less than 500 Da and 16 peptides greater than 3500 Da (one as large as 10,266 Da). Trypsin, in contrast, yielded 106 peptide fragments, with 25 peptides less than 500 Da, and only 4 peptides over 3500 Da. Thus, half as many peptides overall, two-fold fewer peptides of less than 500 Da, would be generated using Lys-C relative to trypsin digestion.

EGFR (1 pmole) was digested with Lys-C and then analyzed by the method of the invention using several injections of 200 fmole each onto a 75 μm i.d. Biobasic C-4 column (300 Å pore). A total of 95% sequence coverage of EGFR, including the identification and location of 10 glycosylation sites and three phosphorylation sites, was found (see Table 2). In the following, we illustrate key points of this analysis. As in the discussion of the beta-casein analysis, because the identification of unmodified peptides less than 3500 Da was similar to conventional methods, we present details of analysis of some of the large peptides: one unmodified, two phosphorylated, and two glycosylated.

TABLE 2
Peptides identified according to the invention^{a} - Epidermal Growth Factor Receptor

| | | | [M + H]⁺ | | |
|---|---|---|---|---|---|
| start | end | Peptide Sequence^{b} | (Da)^{c} | Identification^{d} | Charge States |
| 1 | 4 | LEEK | 518.2820 | − | − |
| 5 | 5 | K | 147.1128 | − | − |
| 6 | 13 | VCQGTSNK | 893.4145 | + | 2+ |
| 14 | 56 | LTQLGTFEDHFLSLQRMFN*NCEVVL | 5165.5863 | +^{e} | 5+ |
| | | GNLEITYVQRNYDLSFLK | | | |
| 57 | 105 | TIQEVAGYVLIALNTVERIPLENLQI | 5559.8831 | +^{g} | 4+, 5+ |
| | | IRGNMYYENSYALAVLSNYDAN*K | | | |
| 106 | 109 | TGLK | 418.2660 | − | − |
| 110 | 165 | ELPMRNLQEILHGAVRFSNNPALCNV | 6533.1104 | +^{f} | 6+, 7+ |
| | | ESIQWRDIVSSDFLSN*MSMDFQNHL | | | |
| | | GSCQK | | | |
| 166 | 185 | CDPSCPN*GSCWGAGEENCQK | 2312.8423 | +^{g} | 2+, 3+ |
| 186 | 188 | LTK | 361.2445 | − | − |
| 189 | 202 | IICAQQCSGRCRGK | 1693.8043 | + | 2+ |
| 203 | 229 | SPSDCCHNQCAAGCTGPRESDCLVCR | 3182.2747 | + | 2+, 3+, 4+, 5+ |
| | | K | | | |
| 230 | 237 | FRDEATCK | 1026.4673 | + | 1+, 2+ |
| 238 | 260 | DTCPPLMLYNPTTYQMDVNPEGK | 2684.2041 | + | 2+, 3+ |
| 261 | 269 | YSFGATCVK | 1032.4819 | + | 1+, 2+ |
| 270 | 301 | KCPRNYVVTDHGSCVRACGADSYEME | 3745.6573 | + | 4+, 5+, 6+ |
| | | EDGVRK | | | |
| 271 | 301 | CPRNYVVTDHGSCVRACGADSYEMEE | 3617.5624 | + | 4+, 5+, 6+ |
| | | DGVRK | | | |
| 302 | 303 | CK | 307.1434 | − | − |
| 304 | 304 | K | 147.1128 | − | − |
| 305 | 311 | CEGPCRK | 906.3920 | − | − |
| 312 | 322 | VCNGIGIGEFK | 1193.5983 | + | 1+, 2+ |
| 323 | 333 | DSLSIN*ATNIK | 1175.6266 | +^{f} | 1+ |
| 334 | 336 | HFK | 431.2401 | − | − |
| 337 | 372 | N*CTSISGDLHILPVAFRGDSFTHTP | 4004.0221 | +^{f} | 3+, 4+, 5+ |
| | | PLDPQELDILK | | | |
| 373 | 375 | TVK | 347.2289 | − | − |
| 376 | 407 | EITGFLLIQAWPEN*RTDLHAFENLE | 3781.0183 | +^{e} | 4+ |
| | | IIRGRTK | | | |
| 408 | 430 | QHGQFSLAVVSLN*ITSLGLRSLK | 2468.3983 | +^{f} | 3+ |
| 431 | 443 | EISDGDVIISGNK | 1346.6798 | + | 1+, 2+ |
| 444 | 454 | NLCYANTINWK | 1396.6678 | + | 1+, 2+ |
| 455 | 463 | KLFGTSGQK | 965.5414 | + | 2+ |
| 456 | 463 | LFGTSGQK | 837.4465 | + | 1+, 2+ |
| 464 | 465 | TK | 248.1605 | − | − |
| 466 | 476 | IISNRGENSCK | 1277.6266 | + | 2+ |
| 477 | 514 | ATGQVCHALCSPEGCWGPEPRDCVSC | 4446.9423 | +^{f} | 4+, 5+, 6+, |
| | | RN*VSRGRECVDK | | | 7+ |
| 515 | 569 | CNLLEGEPREFVENSECIQCHPECLP | 6570.8425 | +^{e} | 5+, 6+, 7+, |
| | | QAMN*ITCTGRGPDNCIQCAHYIDGP | | | 8+ |
| | | HCVK | | | |
| 570 | 585 | TCPAGVMGEN*NTLVWK | 1776.8407 | +^{e} | 2+, 3+ |
| 586 | 618 | YADAGHVCHLCHPN*CTYGCTGPGLE | 3657.5184 | +^{f} | 3+, 4+, 5+ |
| | | GCPTNGPK | | | |
| 619 | 652 | IPSIATGMVGALLLLLVVALGIGLFM | 3684.2535 | + | 5+ |
| | | RRRHIVRK | | | |
| 653 | 684 | RTLRRLLQERELVEPLT*PSGEAPNQ | 3710.1551 | +^{h} | 4+, 5+ |
| | | ALLRILK | | | |
| 685 | 713 | ETEFKKIKVLGSGAFGTVYKVKGLWI | 3211.7401 | + | 4+ |
| | | PEGEK | | | |
| 693 | 704 | VLGSGAFGTVYK | 1198.6466 | + | 1+, 2+ |
| 705 | 713 | GLWIPEGEK | 1028.5411 | + | 1+, 2+ |
| 714 | 721 | VKIPVAIK | 867.6026 | + | 2+ |
| 716 | 721 | IPVAIK | 640.4392 | + | 1+ |
| 722 | 730 | ELREATSPK | 1030.5527 | + | 1+, 2+ |
| 731 | 782 | ANKEILDEAYVMASVDNPHVCRLLGI | 6031.0409 | + | 5+, 6+ |
| | | CLTSTVQLITQLMPFGCLLDYVREHK | | | |
| 734 | 782 | EILDEAYVMASVDNPHVCRLLGICLT | 5717.8660 | + | 4+, 5+, 6+ |
| | | STVQLITQLMPFGCLLDYVREHK | | | |
| 783 | 799 | DNIGSQYLLNWCVQIAK | 2022.0113 | + | 2+ |
| 800 | 822 | GMNYLEDRRLVHRDLAARNVLVK | 2738.4994 | + | 4+ |
| 823 | 828 | TPQHVK | 709.3991 | − | − |
| 829 | 836 | ITDFGLAK | 864.4825 | + | 1+ |
| 837 | 843 | LLGAEEK | 759.4247 | + | 1+ |
| 844 | 855 | EYHAEGGKVPIK | 1327.7004 | + | 2+, 3+ |
| 844 | 905 | EYHAEGGKVPIKWMALESILHRIYTH | 7079.5425 | + | 5+, 6+, 7+, |
| | | QSDVWSYGVTVWELMTFGSKPYDGIP | | | 8+ |
| | | ASEISSILEK | | | |
| 906 | 925 | GERLPQPPICTIDVYMIMVK | 2360.2174 | + | 2+, 3+ |
| 926 | 936 | CWMIDADSRPK | 1378.6242 | + | 2+, 3+ |
| 937 | 946 | FRELIIEFSK | 1281.7201 | + | 2+ |
| 947 | 1037 | MARDPQRYLVIQGDERMHLPS*PTDS | 10266.8256 | +^{h} | 7+, 8+, 9+, |
| | | NFYRALMDEEDMDDVVDADEYLIPQQ | | | 10+, 11+, 12+ |
| | | GFFSS*PSTSRTPLLSSLSATSNNST | | | |
| | | VACIDRNGLQSCPIK | | | |
| 1038 | 1075 | EDSFLQRYSSDPTGALTEDSIDDTFL | 4274.0299 | + | 3+, 4+ |
| | | PVPEYINQSVPK | | | |
| 1076 | 1136 | RPAGSVQNPVYHNQPLNPAPSRDPHY | 6789.2100 | + | 6+ |
| | | QDPHSTAVGNPEYLNTVQPTCVNSTF | | | |
| | | DSPAHWAQK | | | |
| 1137 | 1155 | GSHQISLDNPDYQQDFFPK | 2236.0305 | + | 2+, 3+ |
| 1156 | 1164 | EAKPNGIFK | 1003.5571 | + | 1+ |
| 1165 | 1186 | GSTAENAEYLRVAPQSSEFIGA | 2297.1044 | + | 2+, 3+ |

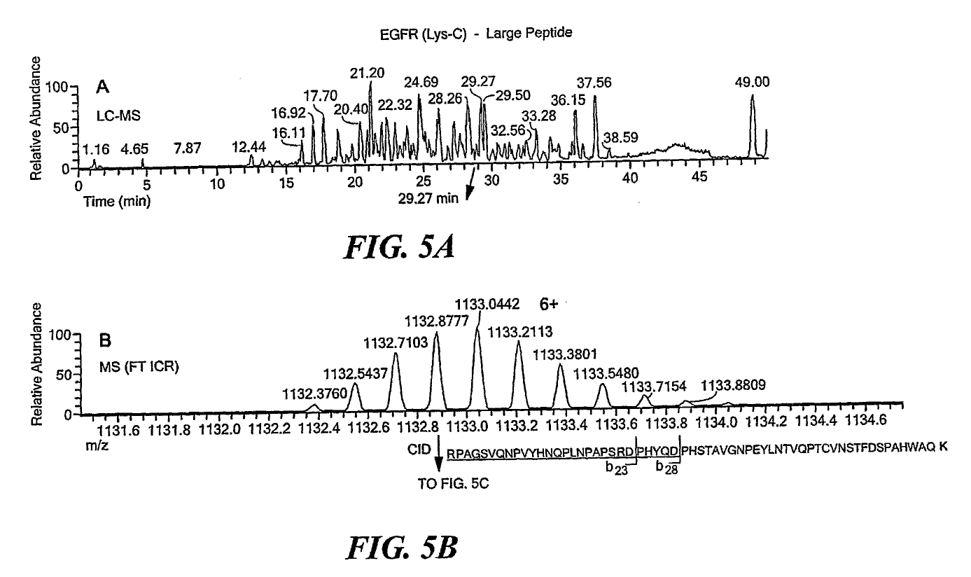

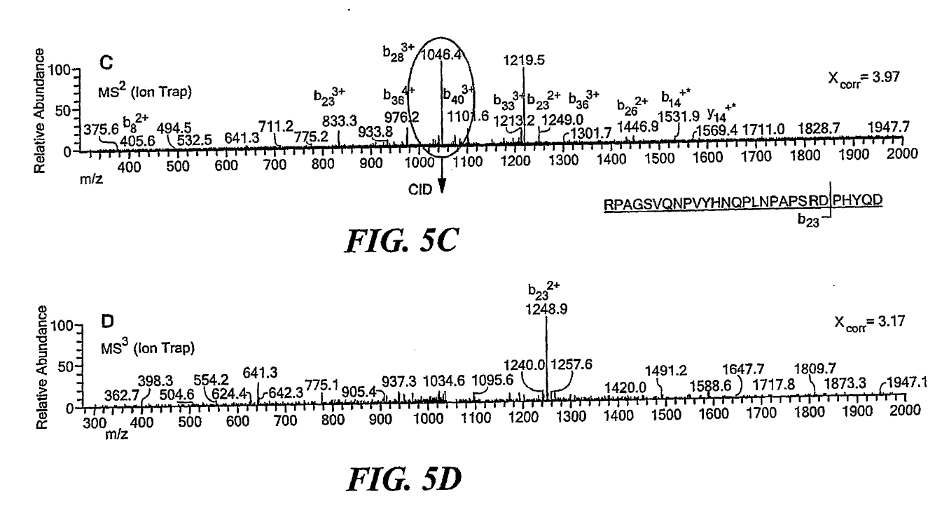

^{a}See FIG. 5 caption and Experimental Section for details.
^{b}“*” indicates glycosylated (N*) or phosphorylated amino acids (S* or T*).
^{c}Theoretical unmodified monoisotope with a hydrogen mass [M + H]⁺shown, including the molecular weight of carbamidomethylation for cysteine residues.
^{d}“+” = identified, “−” = not identified.
^{e}Both glycosylated and nonglycosylated peptides found.
^{f}Only glycosylated peptide found.
^{g}Only nonglycosylated peptide found.
^{h}Both phosphorylated and nonphosphorylated peptides found.

We first examine the analysis of one of the large unmodified peptides. The elution profile of the Lys-C digest of EGFR is shown in the total ion chromatogram of FIG. 5, Panel A. At the elution time of 29.27 min, indicated in FIG. 5, a precursor ion with a charge state of 6+ and a monoisotopic mass m/z of 1132.3760 (6789.2200 Da) was observed in the FTICR survey scan (Panel B). The most intense MS ion was isolated automatically by data-dependent acquisition for subsequent MS² CID fragmentation in the linear ion trap (Panel C), and the most intense MS² ion (m/z=1046.4) isolated for MS³ fragmentation (Panel D). From the MS² spectrum, Sequest determined this large peptide to be EGFR residues 1076-1136 (Panel C, insert). The MS³ spectrum identified a partial sequence of the MS² assignment (1076-1103, underlined). These assignments were further confirmed manually. First, the accurate mass measurement of the precursor ion agreed with the theoretical molecular weight of the candidate peptide within 2 ppm. Second, preferential cleavages were observed among the most intense fragment ions, specifically, cleavage between D and P (b23 and b28 ions), and between N and P (b36 ion), indicated in Panel C. When the b28 ion in the MS² spectrum was further fragmented, the b23 ion was predominant in the MS³ spectrum (Panel D). Again, using both MS² and MS³ preferred fragmentation, together with the accurate mass, strong evidence was provided for the assignment of this 6,789 Da peptide with a 6+ charge state. The MS² and MS³ X_{corr} scores were 3.97 and 3.17, respectively.

It is noteworthy in FIG. 5 that several b ions appear with high intensity in the MS² and MS³ spectra of this Lys-C peptide. Generally, fragments of trypsin, which cleaves at the C-terminal side of both K and R residues, contain these basic residues at the C-terminus of the peptide; hence, only the C-terminal side of the fragmentation point (the y-ions) ionizes well. Because Lys-C fragments may contain internal R residues (two, in the peptide fragmentation of FIG. 5) which provide positive charge on the N-terminal side of the fragmentation bond, Lys-C digestion should improve the chance of observing both y and b ions in a fragmentation spectrum by about 2-fold, relative to a tryptic digest. The observation of both y and b ions in FIG. 5 clearly increases the confidence of the assignments. The other large (unmodified) fragments between 3.5 and 7 kDa were identified by a similar approach, as shown in FIG. 5. All other unmodified peptides in this Lys-C digest of EGFR had predominant charge states of 3+ or lower and were routinely identified by Sequest, as shown in Table 2.

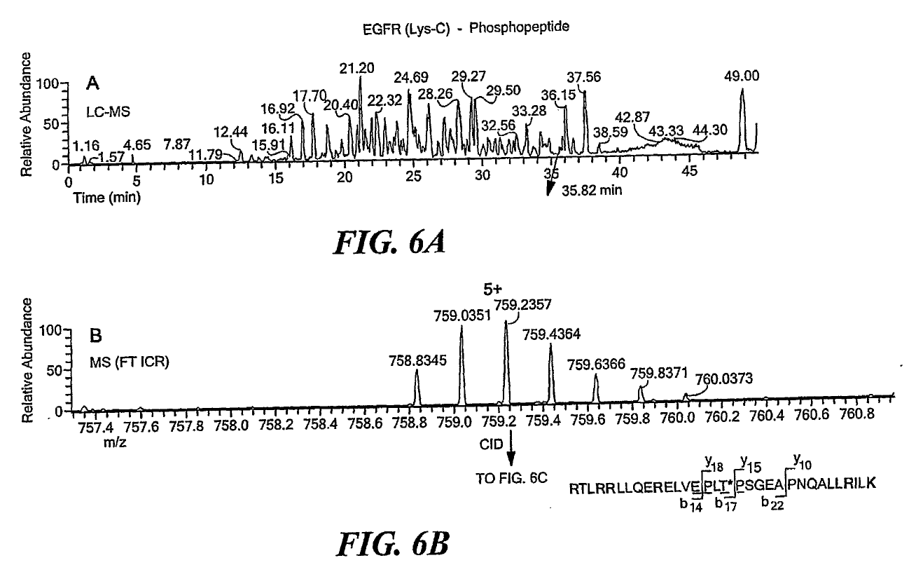

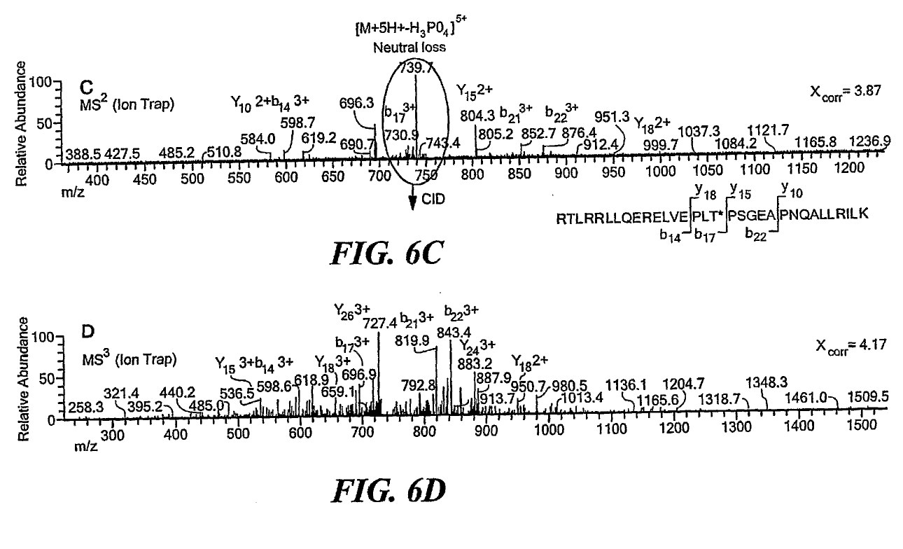

We next turn to an examination of the ability of this method to identify an important phosphorylated peptide in the same Lys-C digest of EGFR. Phosphorylation at T669 has been shown to have a significant effect on intracellular protein kinase activity.⁴¹ At the elution time of 35.82 min, shown in Panel A of FIG. 6, a precursor ion with a charge state of 5+ and a monoisotopic mass m/z of 758.8345 (3790.141 Da) was observed by FTICR MS (Panel B). Similar to the analysis of beta-casein in FIG. 4, MS² CID fragmentation (Panel C), and MS³ fragmentation (Panel D) were automatically acquired in the ion trap by data-dependent acquisition. As shown in FIG. 6, the m/z difference between the most intense ion in the MS² spectrum (Panel C) and its monoisotopic precursor ion in the survey spectrum was 19.6, indicating a phosphate neutral loss of 98 Da for the 5+ charge ion. The site of the phosphorylation was positively identified by the observation of both y18 and y15 ions in the MS² spectrum (insert of Panel C), which determined the site to be T669: the y18 ion (cleavage between E and P, underlined) was observed as a phosphorylated ion, whereas the y15 ion (cleavage between T* and P, underlined) was unmodified. Related fragmentation (both y and b ions) of the peptide with a neutral loss at T669 were also identified in the MS³ spectrum (Panel D), further confirming the assignment. Conclusively, the accurate mass measurement of the precursor ion agreed with the phosphorylated peptide molecular weight within 2 ppm. It is important to note that, in the same analysis at a similar retention time (36.20 min), we also observed the unmodified peptide. The observation of both phosphorylated and unmodified peptides in the same analysis can be used to quantify changes in the degree of phosphorylation at this site upon stimulation of EGFR.

Further examination of FIG. 6 showed significant peptide bond fragmentation in the CID spectrum of the Lys-C phosphopeptide. In contrast, fragmentation of the corresponding tryptic peptide revealed predominantly neutral loss of the phosphate group. In general, tryptic phosphopeptides do not show strong peptide fragmentation in their MS² spectra, so MS³ or higher fragmentation is often needed to assign the peptide sequence. However, in this case the MS² spectrum of the longer Lys-C phosphopeptide (2790 Da) provided sufficient backbone fragmentation to identify the peptide sequence and the presence of phosphorylation (FIG. 6, Panel C). Thus, large phosphopeptides (Ser/Thr phosphorylation) may provide more peptide fragmentation to assign the precursor ion structure in the MS² spectrum than small phosphopeptides.

An important difference was seen between the Lys-C and trypsin digests in the sensitivity of detecting the peptide containing phosphothreonine 669. Loading 200 fmole on the column, we were able to detect the Lys-C phosphopeptide RTLRRLLQERELVEPLT*PSGEAPNQALLRILK (residues 653-684) but could not observe the corresponding tryptic fragment (underlined and in bold) even when loading as much as 2 pmole under similar separation conditions. The tryptic fragment of this phosphopeptide was detected principally as a 2+ charge ion (only 5% as a 3+ ion), while this Lys-C phosphopeptide was principally a 5+ charge ion (with 25% as a 4+ and 25% as a 6+ charge ion). An approximately 30-fold difference in the limit of detection was found between the Lys-C and the corresponding tryptic phosphopeptide ions, when summing the intensities from all observed charge states. Even when comparing only the most intense ion from each series of charges, this difference was still 10- to 15-fold. The improved electrospray ionization efficiency was again attributed to the Lys-C phosphorylated peptide carrying additional R residues, relative to the corresponding tryptic fragment. Moreover, the longer Lys-C peptide eluted 10 minutes later than the corresponding tryptic fragment, and peptides eluting with less aqueous solvent should have improved electrospray ionization efficiency.

We now discuss the analysis of a peptide which was both very large (10,266 Da) as well as phosphorylated (see Table 2). Since Sequest does not support searches for peptides over 10 kDa, manual searching was necessary. For this peptide, we used the accurate precursor mass to narrow down the peptide candidate list. Indeed, employing Lys-C specificity with +5 ppm mass accuracy for such a large molecular weight resulted in only one peptide candidate in the SwissProt database, EGFR residues 947-1037. We then examined the preferred cleavage sites and confirmed that the high intensity ions in the MS² and MS³ spectra were in agreement with the predicted cleavages. A second ion with a mass of 10,346 Da (addition of 80 Da, phosphate) was also observed, and MS² and MS³ fragmentation spectra showed phosphorylation at either S967 or S1002 in this large Lys-C peptide. Thus, this Lys-C fragment was observed in three forms: non-phosphorylated, phosphorylated at S967 or phosphorylated at S1002. Importantly, no doubly-phosphorylated form was observed. The high mass resolution and accuracy of FT-MS was crucial in this analysis to resolve and determine the molecular weight and high charge states of the 10,266 Da peptide.

For additional confirmation of the 10,266 Da peptide assignment, a portion of the Lys-C digest was further cleaved with trypsin. The 10.3 kDa fragment was reduced to seven smaller peptides. Two of the peptides were too small to be analyzed; the other five were identified by Sequest and supported our assignment of the Lys-C fragment. Partial phosphorylation at the same two serine residues was observed in two of these tryptic peptides; but, it could not be determined from the tryptic digest whether the phosphorylation occurred at only one site in a given EGFR molecule, or whether both sites were simultaneously phosphorylated. The accurate mass measurement of the large intact Lys-C peptide (residues 947-1037) did, however, reveal that only one of S967 and S1002 was phosphorylated at any one time. The ability to make this biologically relevant distinction is an important advantage of the method of the invention for large peptide assignment and has the potential to provide a more comprehensive characterization of the protein than tryptic digestion.

With respect to the LC-MS analysis of the Lys-C digest of EGFR, we note that the selection of the capillary LC stationary phase was an important consideration. It is well-known that the recovery of large fragments from LC columns is a function of the pore diameter (and pore shape), along with the n-alkyl chain length.⁴²⁻⁴⁵ In our initial studies to assess recovery, we found that 200 Å or even 300 Å pore commercial C-18 packing materials did not yield full recovery of fragments over 6,000 Da, as evidenced by ghost peaks in subsequent blank runs. In contrast, a C-4, 300 Å pore (Biobasic) solid phase performed well for the separation of the Lys-C digest of EGFR.

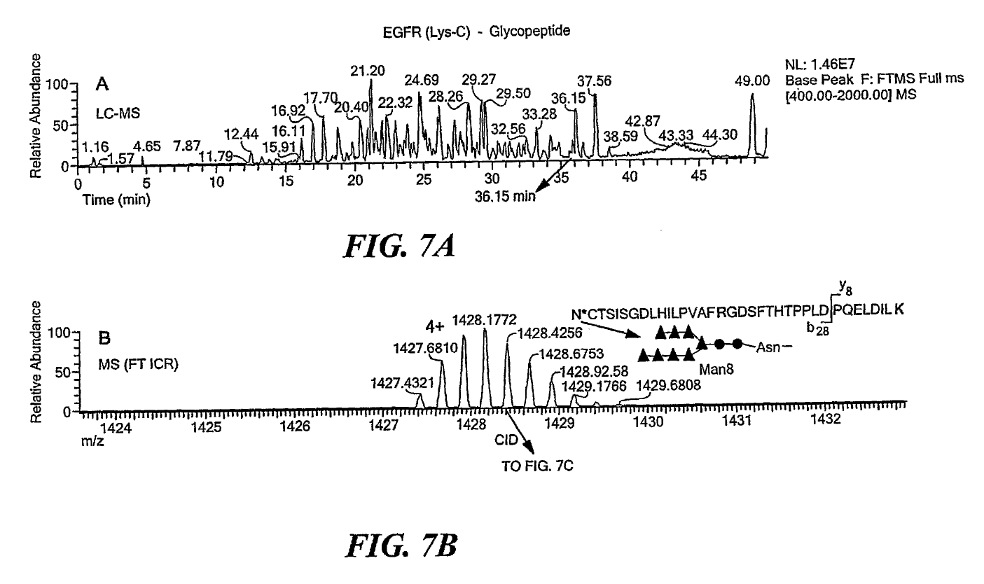

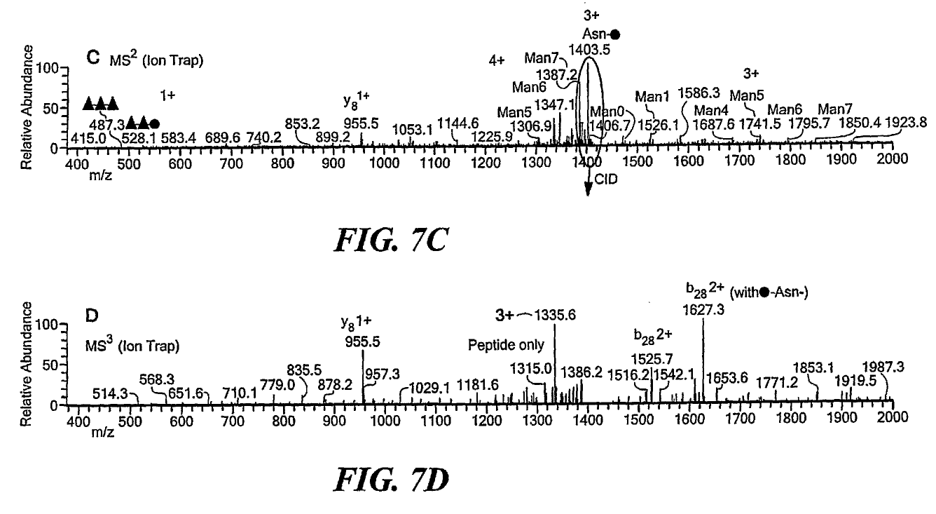

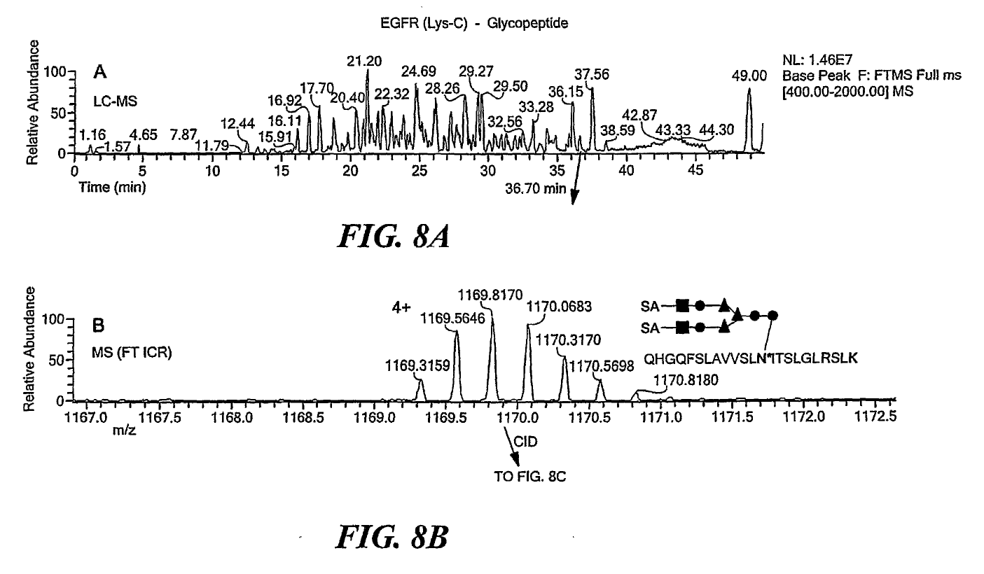

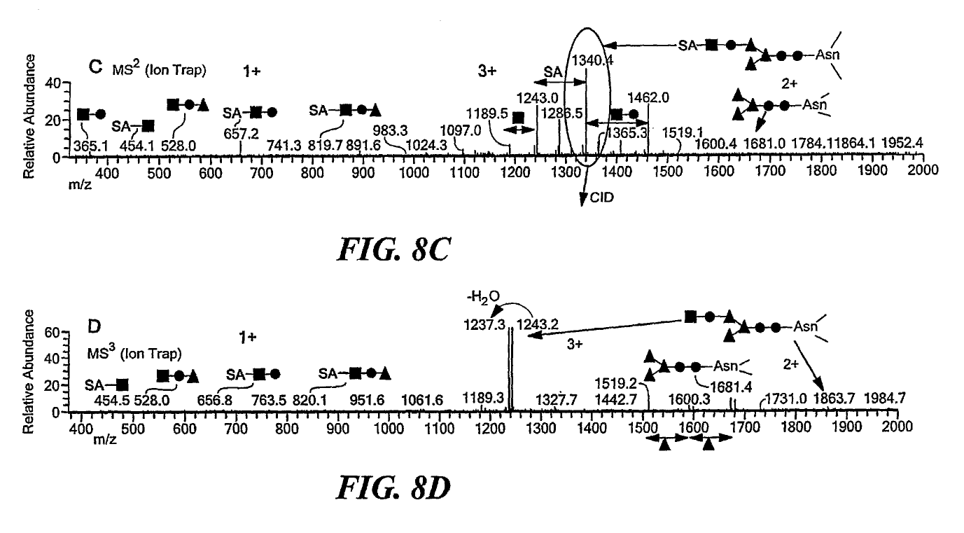

We next examined the capabilities of this strategy in determining the glycosylation pattern of EGFR. In brief, using a total of one pmole of EGFR (200 fmole/injection), we were able to determine which of the 12 potential N-glycosylation sites were fully glycosylated (N151, N328, N337, N420, N504, and N559), partially glycosylated (N32, N389, N544, and N579) or unglycosylated (N104 and N172) (see Table 2). Ongoing characterization of these glycan structures indicates that most are high-mannose or complex-type and so, as representative examples, we will illustrate the analysis of a high-mannose-type glycan and a complex-type glycan in the following discussions of FIGS. 7 and 8.

In contrast to assigning peptides or phosphopeptides, which can be aided by an automated Sequest search, software is limited for recognizing glycopeptides, and therefore the mass spectra at present had to be inspected manually. The search was based on several criteria. Initially, we examined the MS, MS² and MS³ spectra for characteristic features, such as a ladder of fragments differing by the mass of a hexose unit (Hex, Δm=162), typically encountered in the high-mannose type glycans, or for the loss of sialic acid (SA, Δm=292) or N-acetylhexosamine (HexNAc, Δm=203), typically observed for complex-type glycans. In this context, we searched for ladders of masses with charge states as high as the charge state of the precursor ions. In addition, we examined the MS² and MS³ spectra for ions corresponding to the loss of specific oligosaccharide chains, such as antennae (Hex-Hex-Hex, m/z=486; Hex-Hex-HexNAc, m/z=528; or SA-Hex-HexNAc, m/z=657). Since these signature ions are singly charged, their m/z values would be constant regardless of the precursor ion charge state, and therefore, they could be easy to recognize in a spectrum. After a glycopeptide was found by one of the above signatures, the peptide sequence was manually assigned, using one of the strategies discussed in the examples below.

As shown in FIG. 7, a glycopeptide was found at an elution time of 36.15 min (Panel A) with a charge state of 4+ and a monoisotopic mass m/z of 1427.4321 (5706.7066 Da) in the FTMS survey scan (Panel B). The subsequent data-dependent MS² and MS³ CID fragmentation of this precursor ion are shown in Panels C and D. The most likely glycan composition was first deduced from the accurate mass difference between the intact glycopeptide (Panel B) and the unmodified peptide. The unmodified peptide mass was determined, in this case, by comparing the observed precursor ion mass with the predicted masses of the 12 Lys-C peptides with reported N-linked glycosylation sites, each added to the exact molecular weight of the proposed glycan composition (Man 8 in this example). This candidate glycopeptide sequence was then confirmed by the fragmentation in the MS² and MS³ spectra (Panels C and D). Specifically, sequential loss of mannose was observed as Man7 (m/z 1387.2), Man6 (m/z 1347.1), and Man 5 (m/z 1306.9), each with a 4+ charge state (Panel C). The highest intensity ion in MS² (peptide with a single N-acetyglucosamine, m/z 1403.5) was further fragmented to provide the sequence of the peptide portion in the MS³ spectrum (Panel D). Here, we observed that CID produced significant peptide bond fragmentation as well as the glycan cleavage ions. The observation of the deglycosylated peptide fragments in the spectrum facilitated confirmation of the deduced glycan structure, because it enabled the exact molecular weight difference between the glycosylated and deglycosylated peptide to be calculated. Additionally, in the same chromatographic region, we also observed precursor ions with masses corresponding to mannose-5, mannose-6, mannose-7, and mannose-9 attached to the same peptide sequence.

The analysis in FIG. 7 showed a 10-fold greater sensitivity for the Lys-C N-linked glycopeptide fragment, N*CTSISGDLHILPVAFRGDSFTHTPPLDPQELDILK over the corresponding tryptic fragment (underlined in bold) which is attributed to both the additional K residue carried by the Lys-C glycopeptide and the increased hydrophobicity of the larger Lys-C peptide; this glycopeptide eluted much later in the chromatogram than the tryptic glycopeptide.

The ability to identify the glycan moiety depended critically on the determination of the peptide sequence. If peptide backbone fragments of the glycopeptide were not observed in the MS² or MS³ CID spectrum, then two alternative approaches could be taken. First, a higher stage fragmentation such as MS⁴ or MS⁵ could be employed to determine the peptide sequence. However, more material may be required (e.g., 10 fold) for additional LC-MS analyses to target specific precursor masses and to acquire a sufficient number of precursor ions for MS⁴ or MS⁵ fragmentation. A second and more general approach involves performing a parallel analysis according to the method of the invention of a deglycosylated sample (i.e., further treating an aliquot of the Lys-C digest with PNGase F or A), which was used in the next example.

We next turn to the identification of a complex-type glycan structure. A glycopeptide was found at an elution time of 36.70 min (Panel A in FIG. 8) with a charge state of 4+ and a monoisotopic mass m/z of 1169.3159 (4674.2418 Da) in the FTMS survey scan (Panel B). The subsequent data-dependent MS² and MS³ CID fragmentation of this precursor ion are shown in Panels C and D, with the glycan structures shown in the inserts. The most likely glycan composition was first deduced from the accurate mass difference between the intact glycopeptide (Panel B) and the deglycosylated peptide (from analysis of an aliquot of the Lys-C digestion treated with PNGase F, data not shown). The mass of the glycan suggested a bi-antennary structure with a sialic acid at each terminus, as shown in the insert of Panel B. The MS² and MS³ fragmentation (Panels C and D) confirmed this glycan structure: one antennary ion (m/z 657, SA-▪-, 1+) was observed in the MS² spectrum (Panel C), and the other antennary ion (m/z 657, SA-▪-, 1+) was observed in the subsequent MS³ spectrum (Panel D). Also, sequential losses were observed in MS² fragmentation of: sialic acid (from m/z 1340.4, 3+ to m/z 1243.0, 3+; Δm=292.2 Da observed, 291.2 Da theoretical), hexose, presumably galactose (from m/z 1243.0, 3+ to m/z 1189.5, 3+; Δm=160.5 Da observed, 162.1 Da theoretical) and N-acetylglucosamine (from 1189.5, 3+ to m/z 1681.0, 2+; Δm=205.1 Da observed, 203.2 Da theoretical). Together, these fragmentations strongly suggested that the glycan was indeed the bi-antennary structure shown. As discussed above, the signature ions that correspond to complex-type oligosaccharide chains, such as SA-▪- (m/z 657, 1+) and SA-▪--▴ (m/z 819, 1+), were easily recognized in the spectrum. Additionally, in the same chromatographic region, we observed precursor ions with masses corresponding to minor variations of the complex-type glycan structure shown above, such as ions with one less sialic acid, or with an additional fucose and/or N-acetylglucosamine. As previously, the ERPA method again showed 10-fold greater sensitivity for the Lys-C N-linked glycopeptide QHGQFSLAVWSLN*ITSLGLRSLK relative to the corresponding tryptic fragment (underlined and in bold).

Further practice of the method of the invention can include examination of the deglycosylated digest to obtain the sequences of the peptide portions. Deglycosylation can also provide an unambiguous determination of the glycosylation sites, since a glycosidase converts N to D in the removal of the glycan from the peptide.

The detailed glycan structure analysis of each glycopeptide at this stage can be time-consuming because there is at present only limited software that can unambiguously recognize glycosylated peptides or determine their glycan structures, and therefore, individual spectra must be inspected manually. Determining the elution times of deglycosylated peptides greatly reduced the amount of manual examination, as it was found that the glycopeptide and its corresponding deglycosylated form eluted close to each other. Although interpretation was aided by GlycoMod,⁴⁶ the type and branching of the glycans still required manual interpretation based on likely gas-phase cleavages. The development of software to automate the recognition process would be helpful.

### EXPERIMENTAL

### Reagents

Achromobacter protease I (Lys-C) was obtained from Wako (Richmond, Va.), and trypsin (sequencing grade) was purchased from Promega (Madison, Wis.). The proteins, beta-casein from milk and human epidermal growth factor receptor (EGFR) from an A431 cancer cell line, as well as dithiothreitol (DTT), iodoacetamide (IAA), guanidine hydrochloride and ammonium bicarbonate, were obtained from Sigma-Aldrich (St. Louis, Mo.). Formic acid, acetone and acetonitrile were purchased from Fisher Scientific (Fair Lawn, N.J.), and the HPLC-grade water used in all experiments was from J. T. Baker (Bedford, Mass.).

### Enzymatic Digestion

To beta-casein (1 mg/mL), the endoproteinase Lys-C was added in a 1:100 (w/w) ratio, and incubated for 4 hrs at 37° C. Half of the digest was directly analyzed by LC-MS (see below), and the other half of the sample was further digested by trypsin (1:100 w/w) for an additional 16 hr at 37° C., followed by the LC-MS analysis. EGFR was received as a lyophilized powder containing 500 units of the protein. The powder (˜1 pmole of EGFR) was reconstituted with 200 μL of 6 M guanidine hydrochloride, reduced with 20 mM DTT for 30 min at 37° C. and alkylated with 50 mM of IAA in the dark for 1.5 hr at room temperature. After desalting over a Microcon spin column (10 kDa MWCO; Millipore, Bedford Mass.), the endoproteinase Lys-C (1:100 w/w) was added to digest the protein for 4 hr at 37° C. Digestion was stopped by addition of 1% formic acid. Similar to beta-casein, half of the digest was directly analyzed by LC-MS, and the other half was further digested by trypsin (1:100 w/w) for an additional 16 hr at 37° C., then analyzed by LC-MS. For deglycosylation, a portion of the above Lys-C digest (10 μL) was treated with 0.5 unit of PNGase F at 37° C. for 2 hr. In order to avoid possible interactions between silanol groups in the LC stationary phase and phosphate groups in the phosphopeptides, 1% phosphoric acid was added to all samples prior to the LC-MS analysis.²⁰

### LC-MS

LC-MS experiments were performed on an LTQ-FTMS instrument (Thermo Electron, San Jose, Calif.) with an Ultimate nanoLC pump (Dionex, Mountain View, Calif.), using a reversed phase column (75 μm i.d. ×10 cm, BioBasic C18 or C4, 5 μm particle size, Thermo Electron). The flow rate was 400 nL/min for sample loading and 200 nL/min for separation. Mobile phase A was 0.1% formic acid in water, and mobile phase B was 0.1% formic acid in acetonitrile. A shallow gradient was used for all analyses: (i) 5 minutes at 2% B for sample loading at 400 mL/min, (ii) flow rate lowered to 200 nL/min for 5 minutes, (iii) linear gradient to 65% B over 50 min, then (iv) to 80% B over 10 min, and finally (v) constant 80% B for 10 min.

The ion transfer tube of the linear ion trap was held at 245° C.; the normalized collision energy was 28% for MS² and 20% for MS³; and the spray voltage was set at 2.2 kV. Briefly, the mass spectrometer was operated in the data-dependent mode to switch automatically between MS, MS², and MS³ acquisition. Survey full-scan MS spectra with 2 microscans (m/z 400-2000) were acquired in the FTICR cell with mass resolution of 100,000 at m/z 400 (after accumulation to a target value of 2×10⁶ ions in the linear ion trap), followed by 4 pairs of sequential MS² and MS³ scans (see FIG. 1). Each subsequent MS² CID fragmentation (at a target value of 30,000 ions) was performed on a precursor ion which was isolated using the data-dependent acquisition mode to select automatically ions with sequentially highest intensities from the survey scan, with a ±2.5 m/z isolation width. From this MS² fragmentation spectrum, a subsequent MS³ CID fragmentation (at a target value of 5,000 ions) was performed on a precursor ion which was again isolated using the data-dependent acquisition mode with a ±2.5 m/z isolation width to select automatically ions with the highest intensity from the MS² scan. In this acquisition mode, dynamic exclusion was utilized with no repeat counts, and with an exclusion duration of 60 sec. The total cycle time (1 FTICR survey scan with 2 microscans plus 4 pairs of sequential linear ion trap MS² and MS³ scans) was ˜2.7 sec.

### Peptide Assignment

Peptides and proteins were identified by automated searching of all MS² and MS³ spectra against spectra of theoretical fragmentations of a human proteomic database (SwissProt), using the Sequest algorithm incorporated into the BioWorks software (version 3.1 SR1, Thermo Electron). The Sequest search was conducted with a mass tolerance ±4 Da. Peptide ions (≦3+ ions) were assigned automatically with X_{corr} scores above the following thresholds: >3.75 for 3+ ions, >2.2 for 2+ ions, and >1.9 for 1+ ions; with Lys-C or trypsin specificity, as appropriate, and up to 3 missed cleavages. For larger ions (≧4+ charge), Sequest was used to assign the most likely peptide sequence, then confirmed the assignment manually by (i) comparing the accurate precursor mass in the survey scan with the predicted mass of the candidate peptide, (ii) using combined MS² and MS³ peptide assignments and (iii) confirming preferred fragmentation patterns in the observed MS² and MS³ fragmentation, as discussed in the Results and Discussion Section. For peptide identification by MS³ spectra, no enzyme specificity restriction was applied as long as the parent peptides from the MS² assignments were based on full enzyme specificity. In addition, loss of water (−18 Da) from the C terminus was included as a potential modification in the MS³ spectral search. Glycopeptides were assigned manually as described in above.

### REFERENCES

(1) Link, A. J.; Eng, J.; Schieltz, D. M.; Carmack, E.; Mize, G. J.; Morris, D. R.; Garvik, B. M.; Yates, J. R. 3rd. Direct analysis of protein complexes using mass spectrometry. Nat. Biotechnol. 1999, 17, 676-682.
(2) Ling, V.; Guzzetta, A. W.; Canova-Davis, E.; Stults, J. T.; Hancock, W. S.; Covey, T. R.; Shushan, B. I. Characterization of the tryptic map of recombinant DNA derived tissue plasminogen activator by high-performance liquid chromatography-electrospray ionization mass spectrometry. Anal. Chem. 1991, 63, 2909-2915.
(3) Shen, Y.; Jacobs, J. M.; Camp, D. G. 2nd; Fang, R.; Moore, R. J.; Smith, R. D.; Xiao, W.; Davis, R. W.; Tompkins, R. G. Ultra-high-efficiency strong cation exchange LC/RPLC/MS/MS for high dynamic range characterization of the human plasma proteome. Anal. Chem. 2004, 76, 1134-1144.
(4) Lim, H.; Eng, J.; Yates, J. R. 3rd; Tollaksen, S. L.; Giometti, C. S.; Holden, J. F.; Adams, M. W.; Reich, C. I.; Olsen, G. J.; Hays, L. G. Identification of 2D-gel proteins: a comparison of MALDI/TOF peptide mass mapping to μLC-ESI tandem mass spectrometry. J. Am. Soc. Mass Spectrom. 2003, 14, 957-970.
(5) Mann, M.; Jensen, O. N. Proteomic analysis of post-translational modifications. Nat. Biotechnol. 2003, 21, 255-261.
(6) Kelleher, N. L.; Taylor, S. V.; Grannis, D.; Kinsland, C.; Chiu, H. J.; Begley, T. P.; McLafferty, F. W. Efficient sequence analysis of the six gene products (7-74 kDa) from the Escherichia coli thiamin biosynthetic operon by tandem high-resolution mass spectrometry. Protein Sci. 1998, 7, 1796-1801.
(7) McLafferty, F. W.; Fridriksson, E. K.; Horn, D. M.; Lewis, M. A.; Zubarev, R. A. Techview: biochemistry. Biomolecule mass spectrometry. Science 1999, 284, 1289-1290.
(8) Fenn, J. B.; Mann, M.; Meng, C. K.; Wong, S. F.; Whitehouse, C. M. Electrospray ionization for mass spectrometry of large biomolecules. Science 1989, 246, 64-71.
(9) Meng, F.; Forbes, A. J.; Miller, L. M.; Kelleher, N. L. Detection and localization of protein modifications by high resolution tandem mass spectrometry. Mass Spectrom. Rev. 2005, 24, 126-134.
(10) Meng, F.; Du, Y.; Miller, L. M.; Patrie, S. M.; Robinson, D. E.; Kelleher, N. L. Molecular-level description of proteins from saccharomyces cerevisiae using quadrupole FT hybrid mass spectrometry for top down proteomics. Anal. Chem. 2004, 76, 2852-2858.
(11) Hakansson, K.; Chalmers, M. J.; Quinn, J. P.; McFarland, M. A.; Hendrickson, C. L.; Marshall, A. G. Combined electron capture and infrared multiphoton dissociation for multistage MS/MS in a Fourier transform ion cyclotron resonance mass spectrometer. Anal. Chem. 2003, 75, 3256-3262.
(12) Godovac-Zimmermann, J.; Kleiner, O.; Brown, L. R.; Drukier, A. K. Perspectives in spicing up proteomics with splicing. Proteomics 2005, 5, 699-709.
(13) Ge, Y.; Lawhorn, B. G.; ElNaggar, M.; Sze, S. K.; Begley, T. P.; McLafferty, F. W. Detection of four oxidation sites in viral prolyl-4-hydroxylase by top-down mass spectrometry. Protein Sci. 2003, 12, 2320-2326.
(14) Sze, S. K.; Ge, Y.; Oh, H.; McLafferty, F. W. Top-down mass spectrometry of a 29-kDa protein for characterization of any posttranslational modification to within one residue. Proc. Natl. Acad. Sci. U.S.A. 2002, 99, 1774-1779.
(15) Kelleher, N. L.; Lin, H. Y.; Valaskovic, G. A.; Aaserud, D. J.; Fridriksson, E. K.; McLafferty, F. W. Top Down versus Bottom Up Protein Characterization by Tandem High-Resolution Mass Spectrometry. J. Am. Chem. Soc. 1999, 121, 806-812.
(16) Kelleher, N. L.; Zubarev, R. A.; Bush, K.; Furie, B. Furie, B. C.; McLafferty, F. W.; Walsh, C. T. Localization of labile posttranslational modifications by electron capture dissociation: the case of gamma-carboxyglutamic acid. Anal. Chem. 1999, 71, 4250-4253.
(17) Wu, S. L.; Jardine, I.; Hancock, W. S.; Karger, B. L. A new and sensitive on-line liquid chromatography/mass spectrometric approach for top-down protein analysis: the comprehensive analysis of human growth hormone in an E. coli lysate using a hybrid linear ion trap/Fourier transform ion cyclotron resonance mass spectrometer. Rapid Commun. Mass Spectrom. 2004, 18, 2201-2207.
(18) Syka, J. E.; Marto, J. A.; Bai, D. L.; Horning, S.; Senko, M. W.; Schwartz, J. C.; Ueberheide, B.; Garcia, B.; Busby, S.; Muratore, T.; Shabanowitz, J.; Hunt, D. F. Novel linear quadrupole ion trap/FT mass spectrometer: performance characterization and use in the comparative analysis of histone H3 post-translational modifications. J. Proteome Res. 2004, 3, 621-626.
(19) Wu, S. L.; Wieghaus, A.; Metelmann-Strupat, W.; Munster, H.; Griep-Raming, J.; Arnott, D. Proteomics:Instrumentation and Automation (Abstract 449). Proceedings of the 51st ASMS Conference on Mass Spectrometry and Allied Topics, Montreal, Canada 2003.
(20) Kim, J.; Camp, D. G. 2nd; Smith, R. D. Improved detection of multi-phosphorylated peptides in the presence of phosphoric acid in liquid chromatography/mass spectrometry. J. Mass Spectrom. 2004, 39, 208-215.
(21) Ficarro, S. B.; McCleland, M. L.; Stukenberg, P. T.; Burke, D. J.; Ross, M. M.; Shabanowitz, J.; Hunt, D. F.; White, F. M. Phosphoproteome analysis by mass spectrometry and its application to Saccharomyces cerevisiae. Nat. Biotechnol. 2002, 20, 301-305.
(22) Hagglund, P.; Bunkenborg, J.; Elortza, F.; Jensen, O. N.; Roepstorff, P. A new strategy for identification of N-glycosylated proteins and unambiguous assignment of their glycosylation sites using HILIC enrichment and partial deglycosylation. J. Proteome Res. 2004, 3, 556-566.
(23) Olsen, J. V.; Ong, S. E.; Mann, M. Trypsin cleaves exclusively C-terminal to arginine and lysine residues. Mol. Cell. Proteomics 2004, 3, 608-614.
(24) Schwartz, J. C.; Senko, M. W.; Syka, J. E. A two-dimensional quadrupole ion trap mass spectrometer. J. Am. Soc. Mass Spectrom. 2002, 13, 659-669.
(25) Olsen, J. V.; Mann, M. Improved peptide identification in proteomics by two consecutive stages of mass spectrometric fragmentation. Proc. Natl. Acad. Sci. U.S.A. 2004, 101, 13417-13422.
(26) Neubauer, G.; Mann, M. Mapping of phosphorylation sites of gel-isolated proteins by nanoelectrospray tandem mass spectrometry: potentials and limitations. Anal. Chem. 1999, 71, 235-242.
(27) Cochet, C.; Gill, G. N.; Meisenhelder, J.; Cooper, J. A.; Hunter, T. C-kinase phosphorylates the epidermal growth factor receptor and reduces its epidermal growth factor-stimulated tyrosine protein kinase activity. J. Biol. Chem. 1984, 259, 2553-2558.
(28) Davis, R. J.; Czech, M. P. Tumor-promoting phorbol diesters cause the phosphorylation of epidermal growth factor receptors in normal human fibroblasts at threonine-654. Proc. Natl. Acad. Sci. U.S.A. 1985, 82, 1974-1978.
(29) Gamou, S.; Shimizu, N. Calphostin-C stimulates epidermal growth factor receptor phosphorylation and internalization via light-dependent mechanism. J. Cell. Physiol. 1994, 158, 151-159.
(30) http://au.expasy.org/cgi-bin/niceprot.pl?EGFR_HUMAN.
(31) Biscardi, J. S.; Ishizawar, R. C.; Silva, C. M.; Parsons, S. J. Tyrosine kinase signalling in breast cancer: epidermal growth factor receptor and c-Src interactions in breast cancer. Breast Cancer Res. 2000, 2, 203-210.
(32) Laskin, J. J.; Sandler, A. B. Epidermal growth factor receptor: a promising target in solid tumours. Cancer Treat. Rev. 2004, 30, 1-17.
(33) Abd El-Rehim, D. M.; Pinder, S. E.; Paish, C. E.; Bell, J. A.; Rampaul, R. S.; Blamey, R. W.; Robertson, J. F.; Nicholson, R. I.; Ellis, I. O. Expression and co-expression of the members of the epidermal growth factor receptor (EGFR) family in invasive breast carcinoma. Br. J. Cancer. 2004, 91, 1532-1542.
(34) Saez-Valero, J.; Fodero, L. R.; Sjogren, M.; Andreasen, N.; Amici, S.; Gallai, V.; Vanderstichele, H.; Vanmechelen, E.; Parnetti, L.; Blennow, K.; Small, D. H. Glycosylation of acetylcholinesterase and butyrylcholinesterase changes as a function of the duration of Alzheimer's disease. J. Neurosci. Res. 2003, 72, 520-526.
(35) Ashcroft, M.; Kubbutat, M. H.; Vousden, K. H. Regulation of p53 function and stability by phosphorylation. Mol. Cell. Biol. 1999, 19, 1751-1758.
(36) Guo, L.; Kozlosky, C. J.; Ericsson, L. H.; Daniel, T. O.; Cerretti, D. P.; Johnson, R. S. Studies of ligand-induced site-specific phosphorylation of epidermal growth factor receptor. J. Am. Soc. Mass Spectrom. 2003, 14, 1022-1031.
(37) Heisermann, G. J.; Gill, G. N. Epidermal growth factor receptor threonine and serine residues phosphorylated in vivo. J. Biol. Chem. 1988, 263, 13152-13158.
(38) Thelemann, A.; Petti, F.; Griffin, G.; Iwata, K.; Hunt, T.; Settinari, T.; Fenyo, D.; Gibson, N.; Haley, J. D. Phosphotyrosine Signaling Networks in Epidermal Growth Factor Receptor Overexpressing Squamous Carcinoma Cells. Mol. Cell. Proteomics 2005, 4, 356-376.
(39) Zhen, Y.; Caprioli, R. M.; Staros, J. V. Characterization of glycosylation sites of the epidermal growth factor receptor. Biochemistry 2003, 42, 5478-5492.
(40) Forbes, A. J.; Mazur, M. T.; Patel, H. M.; Walsh, C. T.; Kelleher, N. L. Toward efficient analysis of >70 kDa proteins with 100% sequence coverage. Proteomics 2001, 1, 927-933.
(41) Countaway, J. L.; Northwood, I. C.; Davis, R. J. Mechanism of phosphorylation of the epidermal growth factor receptor at threonine 669. J. Biol. Chem. 1989, 264, 10828-10835.
(42) Lewis, R. V.; Fallon, A.; Stein, S.; Gibson, K. D.; Udenfriend, S. Supports for reverse-phase high-performance liquid chromatography of large proteins. Anal. Biochem. 1980, 104, 153-159.
(43) Wilson, Kenneth J.; Van Wieringen, Erika; Klauser, Stefan; Berchtold, Martin W.; Hughes, Graham J. Comparison of the high-performance liquid chromatography of peptides and proteins on 100- and 300-A reversed-phase supports. J. Chromatogr. A 1982, 237, 407-416.
(44) Pearson, J. D.; Regnier, F. E. The Influence of Reversed-Phase Normal-Alkyl Chain-Length on Protein Retention, Resolution, and Recovery—Implications for Preparative HPLC. J. Liq. Chromatogr. 1983, 6, 497-510.
(45) Pearson, J. D.; Mahoney, W. C.; Hermodson, M. A.; Regnier, F. E. Reversed-phase supports for the resolution of large denatured protein fragments. J. Chromatogr. 1981, 207, 325-332.
(46) Cooper, C. A.; Gasteiger, E.; Packer, N. H. GlycoMod—a software tool for determining glycosylation compositions from mass spectrometric data. Proteomics 2001, 1, 340-349.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method of protein characterization comprising the steps of:
providing an aliquot of a sample comprising a protein or a mixture of proteins whose identity is to be determined;
carrying out digestion of said protein or mixture of proteins in said aliquot so that said digestion product comprises at least one fragment having a peptide backbone sequence of greater than or equal to 3000 Da in mass;
separating said digestion product; and
analyzing the structure of one or more of said at least one fragments by mass spectrometry using a mass spectrometer system comprising a mass spectrometer having a mass resolution of at least 25,000 and a mass spectrometer having an electron multiplier detector.

2. The method of claim 1, wherein said separating and analyzing steps are coupled on-line.

3. The method of claim 1, wherein, in said analyzing step, said mass spectrometer system comprises a mass spectrometer having a mass resolution of at least 50,000.

4. The method of claim 1, wherein said mass spectrometer system comprises separate mass spectrometer instruments.

5. The method of claim 1, wherein said mass spectrometer system comprises a single mass spectrometer instrument.

6. The method of claim 1, wherein said mass spectrometer system comprises a hybrid mass spectrometer instrument.

7. The method of claim 1, wherein said digestion is proteolytic enzyme digestion.

8. The method of claim 7, wherein the proteolytic enzyme is selected from the group consisting of Lys-C, Arg-C and Asp-N.

9. The method of claim 7, wherein the proteolytic enzyme digestion is limited digestion with trypsin or Glu-C.

10. The method of claim 1, wherein said digestion is by chemical reaction.

11. The method of claim 10, wherein said chemical reaction is brought about by a compound selected from the group consisting of dilute acid, cyanogen bromide and hydroxylamine.

12. The method of claim 1, wherein said at least one fragment has a peptide backbone sequence of greater than or equal to 4000 Da in mass.

13. The method of claim 1, wherein in said digestion product, greater than 90% of the peptide backbone sequence of said protein or proteins is contained in fragments that are between 500 and 25,000 Da in mass.

14. The method of claim 1, wherein in said digestion product, greater than 90% of the peptide backbone sequence of said protein or proteins is contained in fragments that are between 1000 and 10,000 Da in mass.

15. The method of claim 1, wherein the mass spectrometer having an electron multiplier detector is an ion trap or quadrupole mass spectrometer and the mass spectrometer with a mass resolution of at least 25,000 is a Fourier transform mass spectrometer or a time-of-flight mass spectrometer.

16. The method of claim 15, wherein the mass spectrometer system is a hybrid mass spectrometer that couples an ion trap with a Fourier transform ion cyclotron resonance cell.

17. The method of claim 15, wherein the mass spectrometer system is a hybrid mass spectrometer that couples a quadrupole mass spectrometer with a time-of-flight mass spectrometer.

18. The method of claim 16, wherein the detectors of said hybrid mass spectrometer are operated in parallel.

19. The method of claim 1, wherein an Orbitrap mass spectrometer is used as the mass spectrometer with a mass resolution of at least 25,000.

20. The method of claim 1, wherein said separating step is carried out using liquid chromatography.

21. The method of claim 1, wherein said separating step is carried out using capillary electrophoresis.

22. The method of claim 1, wherein said separating step is carried out using capillary electrochromatography.

23. The method of claim 1, wherein said separating step is carried out on a microfluidic chip.

24. A method of determining the identity of different posttranslationally modified isoforms of a protein, said method comprising the steps of;
providing an aliquot of a sample comprising a protein or mixture of proteins whose posttranslationally modified isoforms are to be determined;
carrying out digestion of said protein in said aliquot so that said digestion product comprises at least one fragment having a peptide backbone sequence of greater than or equal to 3000 Da in mass;
separating said digestion product;
identifying the position of fragments containing the common backbone of said posttranslationally modified isoforms; and
analyzing the structure of said fragments containing the common backbone of said posttranslationally modified isoforms by mass spectrometry using a mass spectrometer system comprising a mass spectrometer having a mass resolution of at least 25,000 and a mass spectrometer having an electron multiplier detector.

25. The method of claim 24, said method further comprising the step of quantitatively determining the level of individualized posttranslationally modified isoforms.

26. The method of claim 24, wherein said posttranslational modification comprises glycosylation.

27. The method of claim 24, wherein said posttranslational modification comprises phosphorylation.

28. The method of claim 24, wherein said posttranslational modification comprises sulfation, acetylation or methylation.

29. The method of claim 26, wherein said glycosylated posttranslational modification is further modified by sulfation and/or phosphorylation.

30. The method of claim 1, wherein said separating and analyzing steps are coupled on-line.

31. The method of claim 1, wherein, in said analyzing step, said mass spectrometer system comprises a mass spectrometer having a mass resolution of at least 50,000.

32. The method of claim 1, wherein said mass spectrometer system comprises separate mass spectrometer instruments.

33. The method of claim 1, wherein said mass spectrometer system comprises a single mass spectrometer instrument.

34. The method of claim 1, wherein said mass spectrometer system comprises a hybrid mass spectrometer instrument.

35. The method of claim 1, wherein said digestion is proteolytic enzyme digestion.

36. The method of claim 35, wherein the proteolytic enzyme is selected from the group consisting of Lys-C, Arg-C and Asp-N.

37. The method of claim 35, wherein the proteolytic enzyme digestion is limited digestion with trypsin or Glu-C.

38. The method of claim 1, wherein said digestion is by chemical reaction.

39. The method of claim 38, wherein said chemical reaction is brought about by a compound selected from the group consisting of dilute acid, cyanogen bromide and hydroxylamine.

40. The method of claim 1, wherein said at least one fragment has a peptide backbone sequence of greater than or equal to 4000 Da in mass.

41. The method of claim 1, wherein in said digestion product, greater than 90% of the peptide backbone sequence of said protein or proteins is contained in fragments that are between 500 and 25,000 Da in mass.

42. The method of claim 1, wherein in said digestion product, greater than 90% of the peptide backbone sequence of said protein or proteins is contained in fragments that are between 1000 and 10,000 Da in mass.

43. The method of claim 1, wherein the mass spectrometer having an electron multiplier detector is an ion trap or quadrupole mass spectrometer and the mass spectrometer with a mass resolution of at least 25,000 is a Fourier transform mass spectrometer or a time-of-flight mass spectrometer.

44. The method of claim 43, wherein the mass spectrometer system is a hybrid mass spectrometer that couples an ion trap with a Fourier transform ion cyclotron resonance cell.

45. The method of claim 43, wherein the mass spectrometer system is a hybrid mass spectrometer that couples a quadrupole mass spectrometer with a time-of-flight mass spectrometer.

46. The method of claim 44, wherein the detectors of said hybrid mass spectrometer are operated in parallel.

47. The method of claim 1, wherein an Orbitrap mass spectrometer is used as the mass spectrometer with a mass resolution of at least 25,000.

48. The method of claim 1, wherein said separating step is carried out using liquid chromatography.

49. The method of claim 1, wherein said separating step is carried out using capillary electrophoresis.

50. The method of claim 1, wherein said separating step is carried out using capillary electrochromatography.

51. The method of claim 1, wherein said separating step is carried out on a microfluidic chip.
